(19) United States
(12) Patent Application Publication
(10) Pub. No.: US 2026/0308196 A1
(43) Pub. Date: Oct. 8, 2026

(54) HELICASE INHIBITORS

(71) Applicant: H. LEE MOFFITT CANCER CENTER AND RESEARCH INSTITUTE, INC., Tampa, FL (US)

(72) Inventors: Mark ALEXANDROW, Lutz, FL (US); Harshani LAWRENCE, Lutz, FL (US); Nicholas LAWRENCE, Lutz, FL (US)

(21) Appl. No.: 19/477,610

(22) PCT Filed: Apr. 22, 2024
(86) PCT No.: PCT/US2024/025658

§ 371 Date: Oct. 22, 2025

Related U.S. Application Data
(60) Provisional application No. 63/461,248, filed on Apr. 22, 2023.

(51) Int. Cl. C07H 17/075; A61K 31/7056; A61K 45/06

(52) U.S. Cl. CPC C07H 17/075; A61K 31/7056; A61K 45/06

(57) ABSTRACT

This disclosure provides compounds useful for the treatment of medical disorders, such as cancers, and more particularly to inhibitors of helicases, such as Superfamily 3 (SF3) and Superfamily 6 (SF6) helicases.

### CROSS-REFERENCE TO RELATED APPLICATIONS

This application claims the benefit of priority to U.S. Provisional Application No. 63/461,248 filed Apr. 22, 2023, the disclosure of which is incorporated herein by reference in its entirety.

### STATEMENT REGARDING FEDERALLY SPONSORED RESEARCH OR DEVELOPMENT

This invention was made with government support under Grant Nos. P30-CA076292, R50CA211447, and GM140140 awarded by the National Institutes of Health. The Government has certain rights in the invention.

### TECHNICAL FIELD

This disclosure relates to compounds for the treatment of medical disorders, such as cancers, and more particularly to inhibitors of helicases, such as Superfamily 3 (SF3) and Superfamily 6 (SF6) helicases.

### BACKGROUND

Helicases are a class of enzymes that unpack an organism's genes. They are motor proteins that move directionally along a nucleic acid phosphodiester backbone, separating two annealed nucleic acid strands using energy from ATP hydrolysis. There are many helicases representing the great variety of processes in which strand separation must be catalyzed, such as DNA replication, transcription, translation, recombination, DNA repair, and ribosome biogenesis. Helicases are classified into six superfamilies based on their shared sequence motifs; helicases not forming a ring structure are in superfamilies 1 and 2, and ring-forming helicases form part of superfamilies 3 to 6. In particular, superfamily 3 (SF3) consists of AAA+ helicases encoded mainly by small DNA viruses and some large nucleocytoplasmic DNA viruses, with the most known being the papilloma virus E1 helicase. Superfamily 6 (SF6) contains the core AAA+ that is not included in the SF3 classification; some proteins in this group are mini chromosome maintenance MCM, CMG, RuvB, RuvA, and RuvC.

The human replicative helicase functions during DNA replication to melt double-stranded DNA (dsDNA), allowing the polymerases and other replisome components access to single-stranded DNA (ssDNA) for synthesis of daughter strands of DNA. The human replicative helicase is referred to as the CMG helicase, which is derived from the names of its core subunits: Cdc45-MCM complex-GINS complex. MCM subunits are the six Mcm2-7 proteins, and GINS subunits are the four proteins named Go-Ichi-Ni-San (Japanese for 5-1-2-3). The entire CMG holoenzyme is thus composed of 11 subunits, Cdc45, 6 MCM monomers, and 4 GINS subunits, producing a very large ~750-800 kDa enzyme. Enzymatic activity of the CMG helicase is derived from ATP binding and hydrolysis within the regions between each pair of MCM dimers that make up the MCM hexamer. There are six pairs of MCMs that form six distinct ATP hydrolysis clefts, which work in a non-symmetrical and combinatorial manner to hydrolyze ATP and alter MCM subunit structures to achieve movement along DNA during the melting steps. ATP hydrolysis alters a staircase structure within the central channel of the MCM hexamer through which ssDNA moves in response to changes to this staircase structure during inter-coordinated ATP hydrolysis steps between MCM subunits.

The CMG helicase is an attractive target for cancer drug development due to unique features of its assembly, utilization in cells, and oncogene-induced errors in CMG management that lead to replication dysfunction n during tumorigenesis and chemotherapeutic intervention. During the cell cycle mammalian cells are ‘smart’ and predict that they will likely encounter problems during the replicative S-phase, when DNA is being duplicated. For this reason, mammalian cells assemble more CMG helicase precursors, the MCM hexamer, than will be required to complete a normal unperturbed S-phase. An excess of reserve MCM hexamers (~5× needed) are pre-loaded onto DNA prior to S-phase beginning. Only a subset of these MCM hexamers are chosen, apparently stochastically, to become full CMG helicases upon recruitment of Cdc45 and GINS subunits. Those CMG helicases that form are active during DNA replication. However, if the cell encounters problems such as fork stalling events due to heterochromatin resistance or chemotherapeutic drug exposure that stall forks, then the excess (reserve) MCM hexamers become converted to functional CMG helicases to recover from the fork stalling events. The previous CMG helicases that had been functioning stop unwinding DNA, and these new CMGs become necessary to complete S-phase and the cell cycle. Herein lies the problem in cancers: tumor cells have been found to lack a proper number of unused reserve MCM hexamers and cannot easily create new CMG helicases as needed, for example, in recovering from chemotherapy drugs.

There are two currently known mechanisms by which tumor cells mismanage MCM/CMG helicases and thus fail to contain enough reserve helicases. First, overexpression of Cyclin E, which is oncogenic for a number of tumor types, leads to a reduction in MCM hexamer loading onto DNA in tumor cells. This results in a lower yield of total MCM hexamers that could become CMGs for replication or recovery (relative to non-tumor cells that load normal levels of MCMs). Second, Myc overexpression, which is known to occur in 70% or more of human malignancies, produces a related but different effect. Myc is known to be involved in stimulating the assembly and activation of CMG helicases (from MCM hexamers), but too much Myc causes this process to become deregulated and leads to excessive CMG helicase activation. This extra activation of CMG helicases by Myc leads to a loss of unused reserve MCMs, as they have already been turned on by the excess Myc proteins. When a tumor cell with excessive Myc and overactive CMGs is exposed to fork stalling chemotherapy, there are not enough unused reserve MCMs available to mount a healthy response to allow survival of the tumor cells. Again, non-tumor cells do not have elevated Myc expression and CMG activation. Therefore, these two known mechanisms by which oncogenes (Cyclin E or Myc) can mismanage MCM/CMG complexes produce a tumor-selective weakness in CMG levels and recovery from replicative stresses such as fork stalling chemotherapy. These findings also argue that a therapeutic window exists between tumor cells and non-tumor cells in a likely poor response of tumor cells to chemotherapy drugs (for example, as combination approaches using a future CMG inhibitor and chemotherapy). It is predicted from these findings that future CMG inhibitors could provide a unique means of cancer intervention for a variety of cancer types since the CMG helicase presents an exploitable tumor-specific vulnerability. Note also that other oncogenes besides Myc and Cyclin E could be found to mismanage CMG dynamics in tumor cells, so this concept could extend beyond just these two examples.

Papillomaviridae is a family of non-enveloped DNA viruses whose members are known as papillomaviruses. Several hundred species of papillomaviruses have been identified, infecting all carefully inspected mammals as well as other vertebrates such as birds, snakes, turtles, and fish. Infection by most papillomavirus types is either asymptomatic or causes small benign tumors, known a papillomas or warts. Papillomas caused by some papillomavirus types carry a risk of becoming cancerous. Papillomaviruses replicate exclusively in the basal layer of the body surface tissues, with all known papillomavirus types infecting a particular body surface, typically the skin or mucosal epithelium of the genitals, anus, mouth, or airways. Papillomaviruses replicate exclusively in keratinocytes, with less-differentiated keratinocyte stem cells thought to be the initial target of productive papillomavirus infections. Subsequent steps in the viral life cycle are strictly dependent on the process of keratinocyte differentiation.

E1, an ATP-dependent DNA helicase, is the only enzyme encoded by papillomaviruses. It is essential for replication and amplification of the viral episome in the nucleus of infected cells. It forms a complex with the viral E2 protein, which is a site-specific DNA-binding transcriptional activator. The E1-E2 complex binds to the replication origin, which contains binding sites for both proteins. In addition to E2, it also interacts with DNA polymerase alpha and replication protein A to effect DNA replication. In solution, E1 is a monomer but binds DNA as a dimer. Recruitment of more E1 subunits to the complex leads to melting of the origin and ultimately to the formation of an E1 hexamer with helicase activity.

Human papillomavirus (HPV) infection is caused by HPV, a DNA virus of the Papillomaviridae family. About 90% of HPV infections cause no symptoms and resolve spontaneously within two years. In some cases, an HPV infection persists and results in either warts or precancerous lesions. These lesions, depending on the site affected, increase the risk of cancer of the cervix, vulva, vagina, penis, anus, mouth, or throat. Over 170 HPV types have been described, with more than 40 able to be spread through sexual contact and infect the anus and genitals. Nearly every individual is infected by HPV at some point in their lives, leading it to be the most common sexually transmitted infection globally.

### SUMMARY

The present disclosure provides compounds that are useful in the treatment of medical disorders. More particularly, compounds are provided which are inhibitors of helicases, such as Superfamily 3 (SF3) and Superfamily 6 (SF6) helicases, which are useful in the treatment of medical disorders such as cancers.

In one aspect, a compound of Formula I is provided

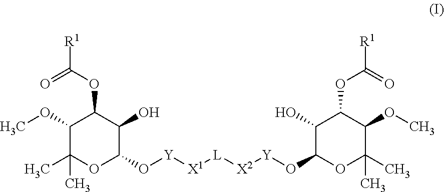

or a pharmaceutically acceptable salt thereof, wherein all variables are as defined herein.

In another aspect, a pharmaceutical composition is provided comprising a compound described herein, or a pharmaceutically acceptable salt thereof, and a pharmaceutically acceptable carrier or excipient.

In another aspect, a method is provided for treating cancer in a subject in need thereof comprising administering to the subject a therapeutically effective amount of a compound described herein, or a pharmaceutically acceptable salt thereof, or a pharmaceutical composition described herein.

In another aspect, a method is provided for treating cancer in a subject in need thereof comprising:
(a) determining whether the cancer is associated with elevated expression of Myc and/or elevated expression of Cyclin E; and
(b) if the cancer is determined to be associated with elevated expression of Myc and/or elevated expression of Cyclin E in (a), administering a therapeutically effective amount of a compound described herein, or a pharmaceutically acceptable salt thereof, or a pharmaceutical composition described herein.

In another aspect, a method is provided for treating cancer in a subject in need thereof, wherein the cancer has been previously determined to be associated with elevated expression of Myc and/or elevated expression of Cyclin E, the method comprising administering a therapeutically effective amount of a compound described herein, or a pharmaceutically acceptable salt thereof, or a pharmaceutical composition described herein.

In another aspect, a method is provided of treating an infection with a papillomavirus in a subject in need thereof comprising administering a therapeutically effective amount of a compound described herein, or a pharmaceutically acceptable salt thereof, or a pharmaceutical composition described herein.

In another aspect, a method is provided for inhibiting and/or degrading a helicase in a eukaryotic cell comprising contacting the cell with an effective amount of a compound described herein, or a pharmaceutically acceptable salt thereof.

In another aspect, a method is provided for inhibiting replication of a papillomavirus in a eukaryotic cell comprising contacting the cell with an effective amount of a compound described herein, or a pharmaceutically acceptable salt thereof.

In another aspect, a method is provided for treating cancer in a subject in need thereof comprising:
(a) determining whether the cancer harbors one or more inherited or acquired germ-line mutations; and
(b) if the cancer is determined to harbor one or more inherited or acquired germ-line mutations in (a), administering to the subject a therapeutically effective amount of a compound described herein, or a pharmaceutically acceptable salt thereof, or a pharmaceutical composition described herein.

In another aspect, a method is provided for treating cancer in a subject in need thereof, wherein the cancer has been previously determined to harbor one or more inherited or acquired germ-line mutations, the method comprising administering to the subject a therapeutically effective amount of a compound described herein, or a pharmaceutically acceptable salt thereof, or a pharmaceutical composition described herein.

In another aspect, a method is provided for treating cancer in a subject in need thereof comprising:
(a) administering to the subject a therapeutically effective amount of a compound described herein, or a pharmaceutically acceptable salt thereof, or a pharmaceutical composition described herein; and
(b) administering an additional therapeutic agent selected from a Chk1 inhibitor, an ATR inhibitor, a Cdc7 inhibitor, and a Parp inhibitor.

The details of one or more aspects of the disclosure are set forth in the accompanying drawings and the description below. Other features, objects, and advantages of the disclosure will be apparent from the description and the claims.

### DESCRIPTION OF DRAWINGS

FIG. 1 is helicase assay (fork-unwinding; strang displacement assay) which shows that RF1-134 inhibits hCMG helixase activity 25% compared to CA1 (100% inhibition). For 20 uL reactions: 1 uL DMSO or drugs, 2 uL hCMG (~15 fmol enzyme), and 0.5 mM ATP were added. The noviose sugar (purple box; called RF1-134) was generated using synthetic chemical approaches. The sugar group alone can partially inhibit the hCMG, versus the complete CA1 compound which inhibits all hMG activity. CA1 refers to coumermycin-A1.

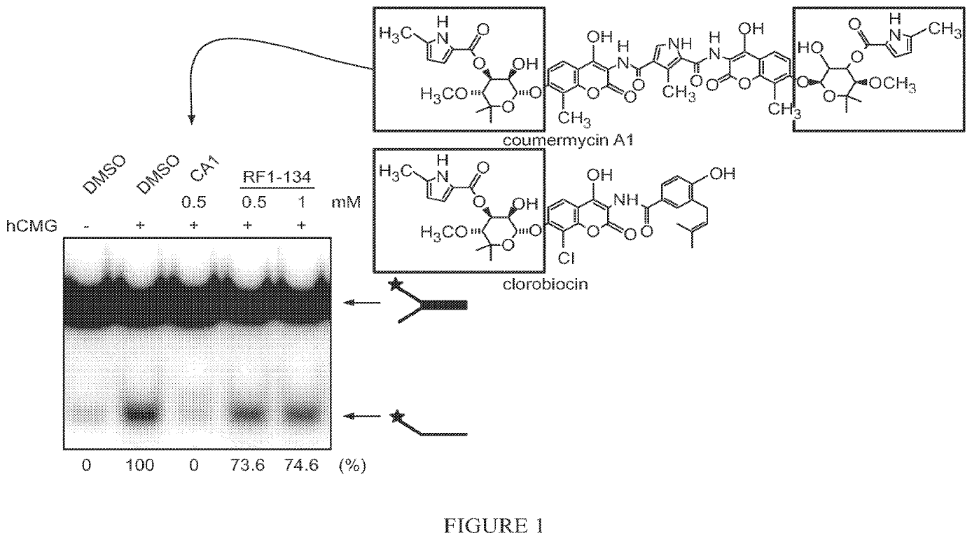

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an,” and “the” include plural referents unless the context clearly dictates otherwise.

It should be noted that ratios, concentrations, amounts, and other numerical data can be expressed herein in a range format. It can be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y’, and ‘about z’ as well as the ranges of ‘less than x’, less than y′, and ‘less than z,’ Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’,” where ‘x’ and ‘y’ are numerical values, includes “about ‘x’ to about ‘y’.”

It is to be understood that such a range format is used for convenience and brevity and, thus, should be interpreted in a flexible manner to include not only the numerical values explicitly recited as the limits of the range but also all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition can also be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for administration purposes. Consequently, single-dose compositions can contain such amounts or submultiples thereof to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons, or virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied, for example, by increasing or decreasing the amount of a disclosed compound and/or pharmaceutical composition, changing the disclosed compound and/or pharmaceutical composition administered, changing the route of administration, changing the dosage timing, and so on. Dosage can vary and can be administered in one or more doses daily for one or several days. Guidance can be found in the literature for appropriate dosages for given classes of pharmaceutical products.

As used herein, the terms “optional” or “optionally” mean that the subsequently described event or circumstance can or cannot occur and that the description includes instances where said event or circumstance occurs and instances where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to humans and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as a cancer. The effect can be therapeutic in terms of a partial or complete cure of a disease, condition, symptom, or adverse effect attributed to the disease, disorder, or condition. The term “treatment” as used herein can include any treatment of a disorder in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment,” as used herein, can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating” can include inhibiting the disease, disorder, or condition, e.g., impeding its progress, and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect or to decreasing the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates, and other isomers, such as rotamers, as if each is specifically described, unless otherwise indicated or otherwise excluded by context. It is to be understood that the compounds provided herein may contain chiral centers. Such chiral centers may be of either the (R—) or (S—) configuration. The compounds provided herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to administration of the compound in its (S—) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Compounds described herein may contain one or more double bonds and, thus, potentially give rise to cis/trans (E/Z) isomers, as well as other conformational isomers. Unless states to the contrary, all such possible isomers are contemplated, as well as mixtures of such isomers.

Compounds described herein may also present as an equilibrium of tautomers. For example, ketones with an α-hydrogen can exist in an equilibrium of the keto form and the enol form. Likewise, amides with an N-hydrogen can exist in an equilibrium of the amide form and the imidic acid form. Unless stated to the contrary, all possible tautomers of the compounds described herein are contemplated.

A dash (“−”) that is not between two letters or symbols is used to indicate a point of attachment for a substituent. For example, —(C═O)NH₂ is attached through the carbon of the keto (C═O) group.

As used herein, the symbol

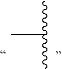

(which hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example

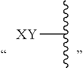

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

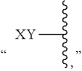

infers that when R³ is “XY,” the point of attachment bond is the same bond as the bond by which R³ is depicted as being bonded to CH₃.

The term “substituted,” as used herein, means that any one or more hydrogens on the designated atom or groups are replaced with a moiety selected from the indicated group, provided that the designated atom's normal valence is not exceeded and the resulting compound is stable. For example, when the substituent is oxo (i.e., ═O), two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and formulated into a dosage form with a shelf life of at least one month. A stable manufacturing intermediate or precursor to an active compound is stable if it does not degrade within the period needed for reaction or other use. A stable moiety or substituent group is one that does not degrade, react, or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the disclosure and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person in the context in which said functional groups are recited.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₄alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl) C₀-C₄alkyl, or —C₀-C₄ (C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one aspect, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one aspect, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one aspect, the alkoxy group is optionally substituted as described herein.

“Alkanoyl” is an alkyl group as defined above covalently bound through a carbonyl (C═O) bridge. The carbonyl carbon is included in the number of carbons, for example, C₂alkanoyl is a CH₃(C═O)— group. In one aspect, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl.

Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3, -dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example, indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring that contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, such as groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one aspect, the total number of S and O atoms in the heteroaryl group is not more than 2. In another aspect, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts that are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH), and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, the term “derivative” refers to a compound having a structure derived from the structure of a parent compound (e.g., a compound disclosed herein) and whose structure is sufficiently similar to those disclosed herein and based upon that similarity would be expected by one skilled in the art to exhibit the same or similar activities and utilities as the claimed compounds, or to induce, as a precursor, the same or similar activities and utilities as the claimed compound. Exemplary derivatives include but are not limited to, salts, esters, amides, salts of esters or amides, and N-oxides of a parent compound.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high-performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers, such as Sigma-Aldrich (formally MilliporeSigma, Burlington, MA) or Thermo Fisher Scientific Inc. (Waltham, MA), or are prepared by methods known to those skilled in the art following procedures set forth in references such as Fieser and Fieser's Reagents for Organic Synthesis (John Wiley and Sons, 2007); Organic Reactions (John Wiley and Sons, 2004); March's Advanced Organic Chemistry, (John Wiley and Sons, 8^{th} Edition); and Larock's Comprehensive Organic Transformations (John Wiley and Sons, 3^{rd} edition, 2017).

### Compounds

The present disclosure provides compounds that may be useful as inhibitors of helicases, such as SF3 and/or SF6 helicases, and more particularly, for example, CMG helicase and HPV E1 helicase. The present compounds have utility in the treatment of medical disorders mediated by an SF3 and/or an SF6 helicase, for example, cancer.

In one aspect, a compound is provided of Formula I:

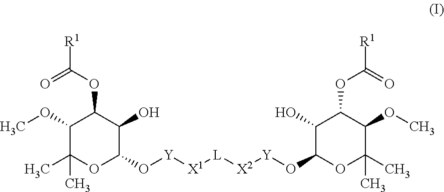

or a pharmaceutically acceptable salt thereof;
wherein:
R¹ is independently selected at each occurrence from monocyclic or bicyclic heteroaryl and monocyclic or bicyclic heterocycloalkyl, wherein each R¹ optionally includes at least one ring nitrogen atom substituted with R⁷ as allowed by valency, and wherein each R¹ is optionally substituted with 1, 2, 3, or 4 groups selected from R⁸ as allowed by valency;
Y is independently selected at each occurrence from a bond, bicyclic aryl, or bicyclic heteroaryl, wherein each Y is optionally substituted with 1, 2, 3, or 4 groups independently selected from R² as allowed by valency;
X¹ is selected from a bond or —NR^{a}—;
L is selected from a bond or -L¹-L²-L³-L⁴-L⁵-L⁶-L⁷-;
L¹, L², L³, L⁴, L⁵, L⁶, and L⁷ are independently selected from:

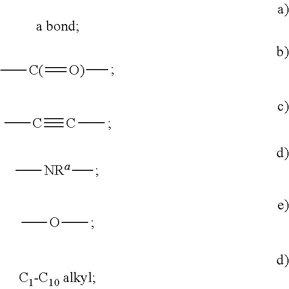

e) cycloalkyl optionally substituted with 1, 2, 3, or 4 substituents independently selected from R³;
f) heterocycloalkyl optionally substituted with 1, 2, 3, or 4 substituents independently selected from R⁴;
g) aryl optionally substituted with 1, 2, 3, or 4 groups independently selected from R⁵;
h) heteroaryl optionally substituted with 1, 2, 3, or 4 groups independently selected from R⁶;

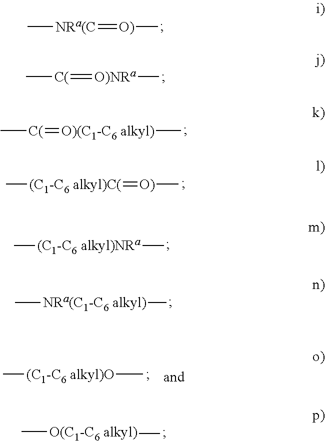

X² is selected from a bond, —NR^{a}—, —O—, —C≡C—, —C(═O)—, —S(═O)₂—, —C(═O)NR^{a}—, —NR^{a}C(═O)—, —S(═O)₂NR^{a}—, —NR^{a}S(═O)₂—, and —NR^{a}(C═O)NR^{b}—;
R² is selected at each occurrence from halogen, hydroxy, nitro, —NHR^{a}, C₁-C₆ alkyl, C₁-C₆ haloalkyl, aryl, and oxo;
R³ and R⁴ are independently selected at each occurrence from halogen, hydroxy, —NHR^{a}, C₁-C₆ alkyl, C₁-C₆ haloalkyl and oxo;
R⁵ and R⁶ are independently selected at each occurrence from halogen, hydroxy, nitro,
   —NHR^{a}, C₁-C₆ alkyl, C₁-C₆ haloalkyl, and aryl;
R⁷ is selected from: hydrogen; C₁-C₄ alkyl; C₂-C₆ alkenyl; C₂-C₆ alkynyl; C₃-C₆ cycloalkyl; C₁-C₆ alkoxy; and (C₀-C₆ alkyl) (aryl) which is optionally substituted with halogen, C₁-C₆ alkyl, or C₁-C₆ alkoxy;
R⁸ is selected from: hydrogen; halogen; hydroxy; —NR^{a}R^{b}; nitro; C₁-C₆ alkyl; C₁-C₆ haloalkyl; C₃-C₆ cycloalkyl; C₃-C₆ heterocycloalkyl; C₂-C₆ alkenyl; C₂-C₆ alkynyl; C₁-C₆ alkoxy; (C₀-C₆ alkyl) (aryl) which is optionally substituted with halogen, C₁-C₆ alkyl, or C₁-C₆ alkoxy; and (C₀-C₆ alkyl) (heteroaryl) which is optionally substituted with halogen, C₁-C₆ alkyl, or C₁-C₆ alkoxy; and
R^{a} and R^{b} are independently selected at each occurrence from hydrogen and C₁-C₆ alkyl.

In some aspects of Formula I, R¹ is

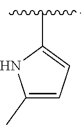 [SMILES: *c1ccc(C)[nH]1]

In some aspects of Formula I, R¹ is selected from:

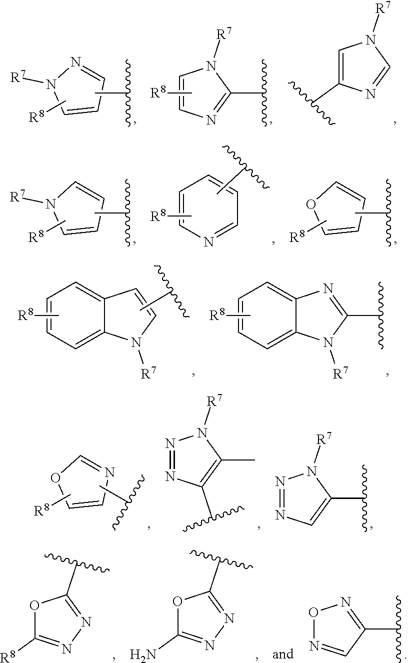

In some aspects of Formula I, R¹ is selected from:

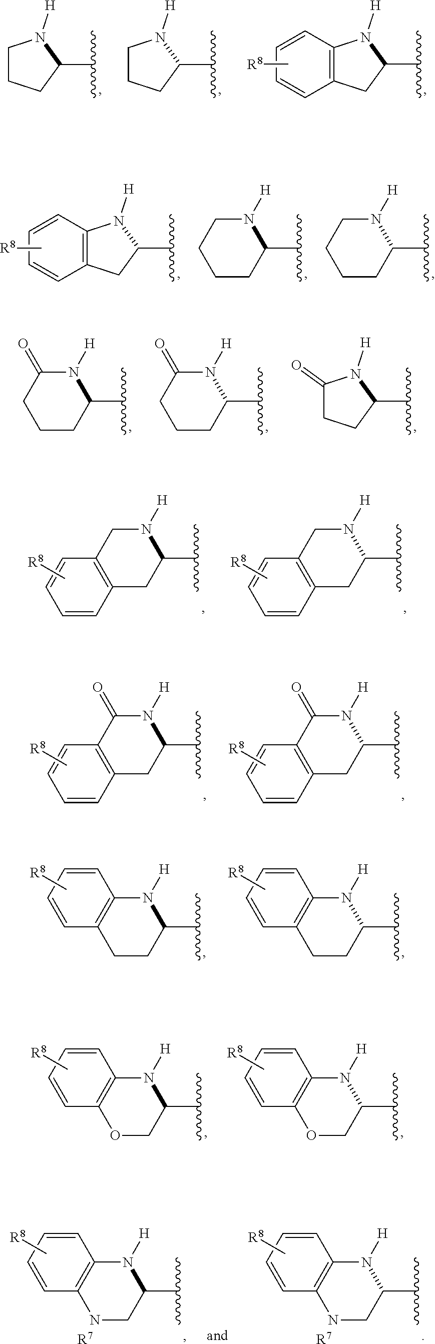

In some aspects of Formula I, Y is a bond.

In some aspects of Formula I, Y is

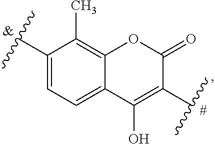 [SMILES: *c1ccc2c(O)c(*)c(=O)oc2c1C]

wherein & denotes the point of attachment to the neighboring oxygen atom and #denotes the point of attachment to X¹ and/or X².

In some aspects of Formula I, Y is selected from:

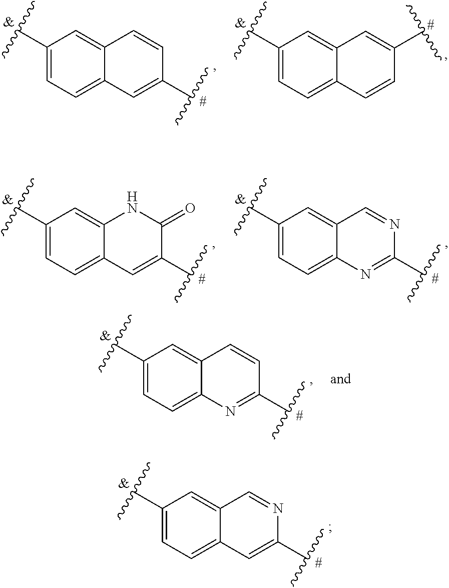 [SMILES: *c1ccc2cc(*)ccc2c1 ; *c1ccc2ccc(*)cc2c1 ; *c1ccc2cc(*)c(=O)[nH]c2c1 ; *c1ccc2nc(*)ncc2c1 ; *c1ccc2nc(*)ccc2c1 ; *c1ccc2cc(*)ncc2c1]

wherein & denotes the point of attachment to the neighboring oxygen atom and #denotes the point of attachment to X¹ and/or X².

In some aspects of Formula I, X¹ is —NH—.

In some aspects of Formula I, L¹, L², L³, L⁴, L⁵, L⁶, and L⁷ are each independently selected from: a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O) NH—; —C(═O) CH₂—; —C(═O) (CH₂)₂—; —C(═O) (CH₂)₃—; —C(═O) (CH₂)₄—; —C(═O) (CH₂)₅—; —C(═O) (CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)₅—; —O(CH₂)₆—;

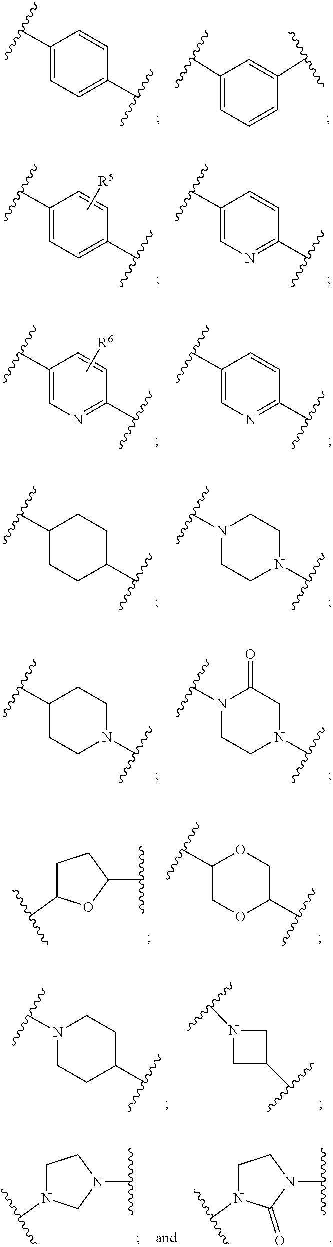

In some aspects of Formula I, L¹, L², L³, L⁴, L⁵, L⁶, and L⁷ are selected in such a way that: no two —C(═O)— moieties are adjected to each other; no two —O— or —NH— moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of L¹, L², L³, L⁴, L⁵, L⁶, and L⁷.

In some aspects of Formula I, L is selected from:

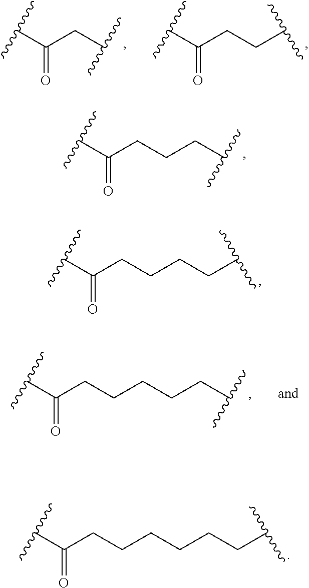 [SMILES: *CC(*)=O ; *CCC(*)=O ; *CCCC(*)=O ; *CCCCC(*)=O ; *CCCCCC(*)=O ; *CCCCCCC(*)=O]

In some aspects of Formula I, L is selected from:

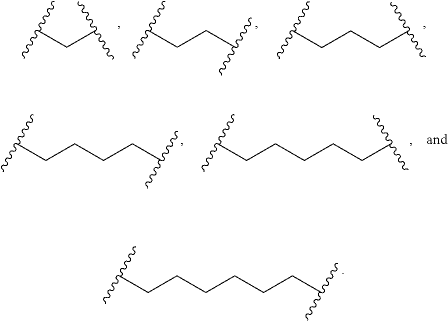

In some aspects of Formula I, L is selected from:

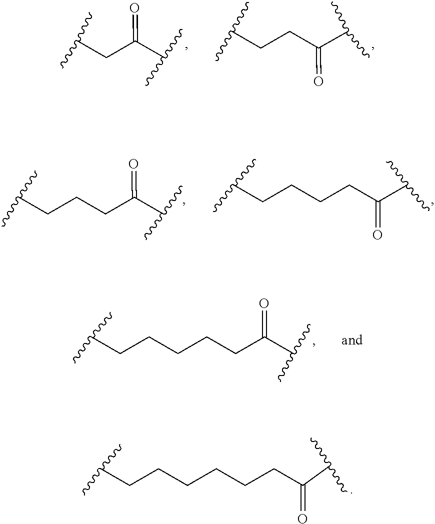 [SMILES: *CC(*)=O ; *CCC(*)=O ; *CCCC(*)=O ; *CCCCC(*)=O ; *CCCCCC(*)=O ; *CCCCCCC(*)=O]

In some aspects of Formula I, L is

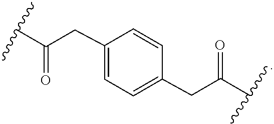 [SMILES: *C(=O)Cc1ccc(CC(*)=O)cc1]

In some aspects of Formula I, L is

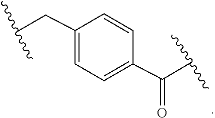 [SMILES: *Cc1ccc(C(*)=O)cc1]

In some aspects of Formula I, L is selected from:

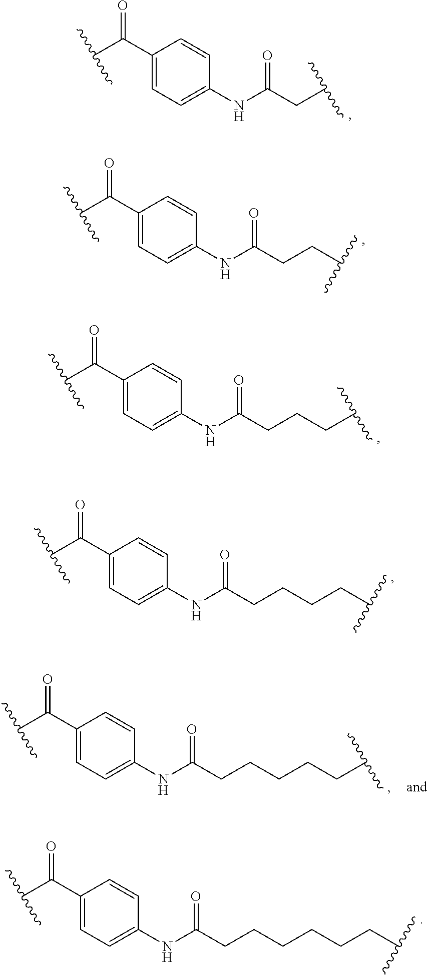 [SMILES: *CC(=O)Nc1ccc(C(*)=O)cc1 ; *CCC(=O)Nc1ccc(C(*)=O)cc1 ; *CCCC(=O)Nc1ccc(C(*)=O)cc1 ; *CCCCC(=O)Nc1ccc(C(*)=O)cc1 ; *CCCCCC(=O)Nc1ccc(C(*)=O)cc1 ; *CCCCCCC(=O)Nc1ccc(C(*)=O)cc1]

In some aspects of Formula I, L is selected from:

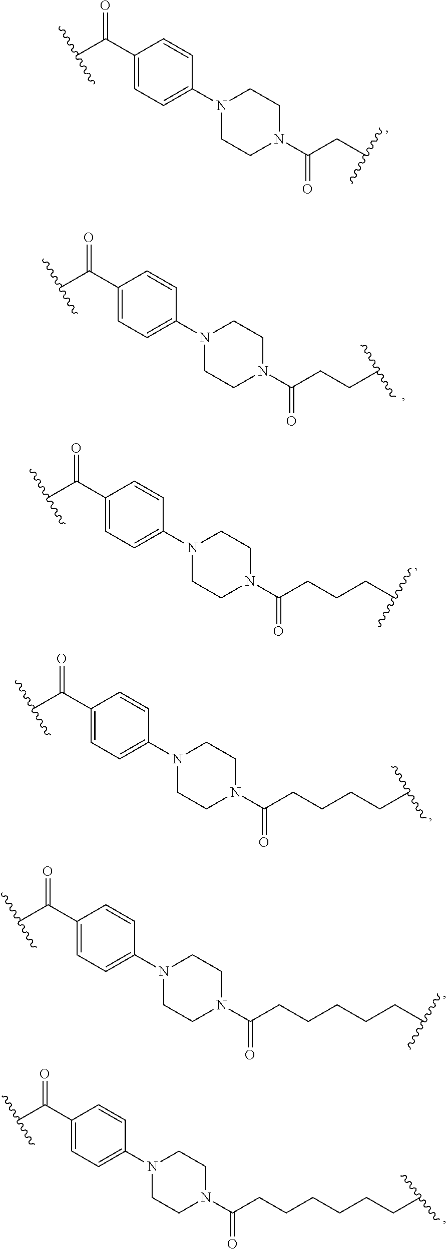 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

In some aspects of Formula I, L is selected from:

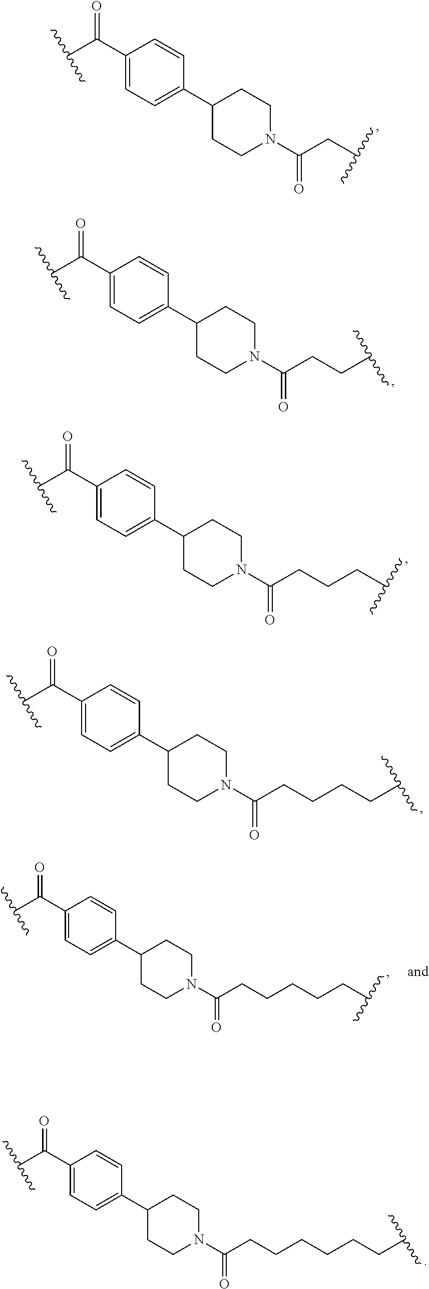 [SMILES: *CC(=O)N1CCC(c2ccc(C(*)=O)cc2)CC1 ; *CCC(=O)N1CCC(c2ccc(C(*)=O)cc2)CC1 ; *CCCC(=O)N1CCC(c2ccc(C(*)=O)cc2)CC1 ; *CCCCC(=O)N1CCC(c2ccc(C(*)=O)cc2)CC1 ; *CCCCCC(=O)N1CCC(c2ccc(C(*)=O)cc2)CC1 ; *CCCCCCC(=O)N1CCC(c2ccc(C(*)=O)cc2)CC1]

In some aspects of Formula I, L is selected from:

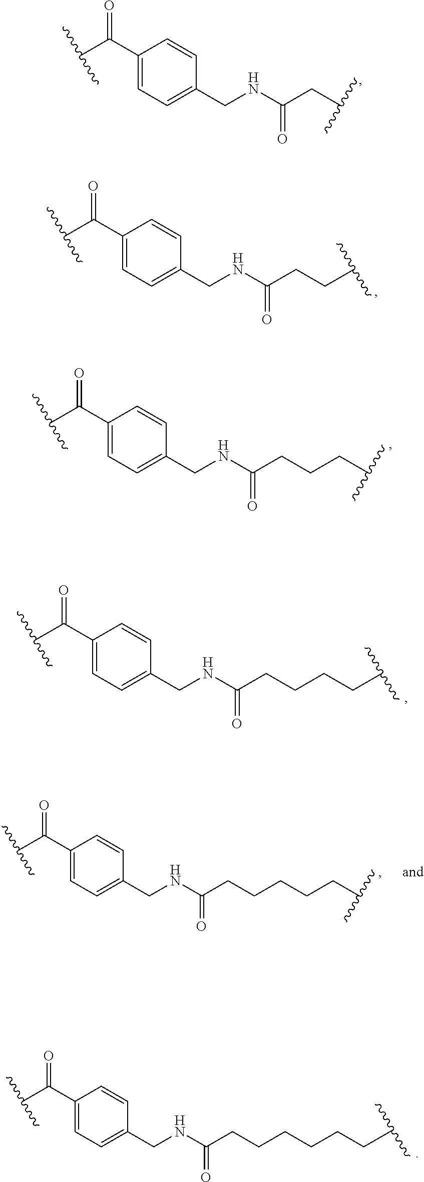 [SMILES: *CC(=O)NCc1ccc(C(*)=O)cc1 ; *CCC(=O)NCc1ccc(C(*)=O)cc1 ; *CCCC(=O)NCc1ccc(C(*)=O)cc1 ; *CCCCC(=O)NCc1ccc(C(*)=O)cc1 ; *CCCCCC(=O)NCc1ccc(C(*)=O)cc1 ; *CCCCCCC(=O)NCc1ccc(C(*)=O)cc1]

In some aspects of Formula I, L is selected from:

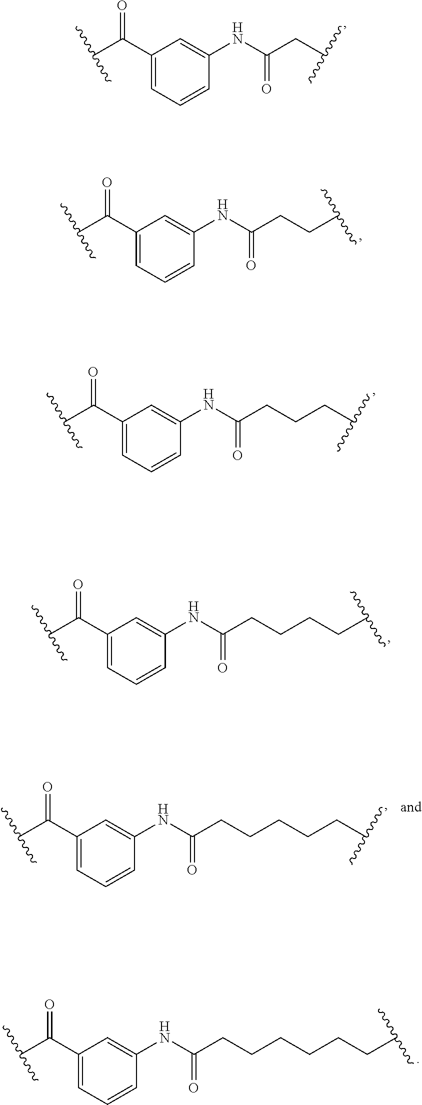 [SMILES: *CC(=O)Nc1cccc(C(*)=O)c1 ; *CCC(=O)Nc1cccc(C(*)=O)c1 ; *CCCC(=O)Nc1cccc(C(*)=O)c1 ; *CCCCC(=O)Nc1cccc(C(*)=O)c1 ; *CCCCCC(=O)Nc1cccc(C(*)=O)c1 ; *CCCCCCC(=O)Nc1cccc(C(*)=O)c1]

In some aspects of Formula I, L is selected from:

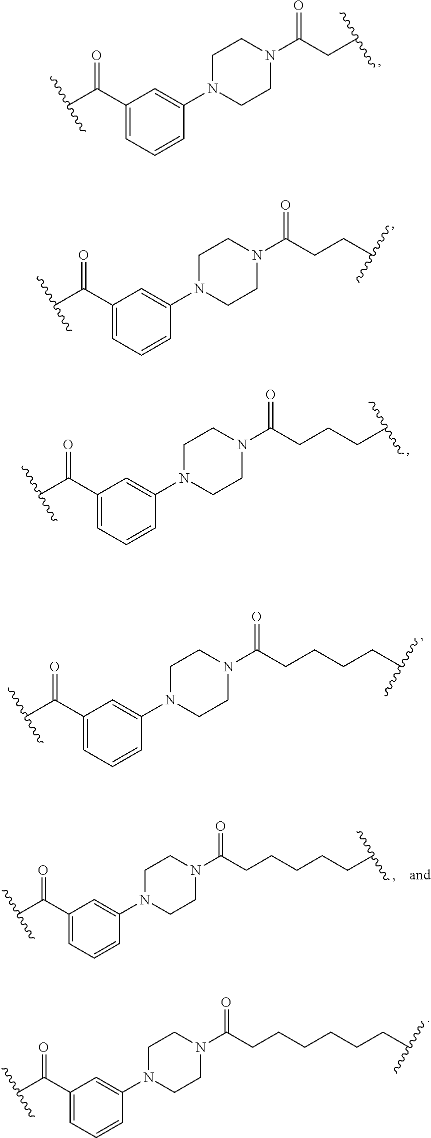 [SMILES: *CC(=O)N1CCN(c2cccc(C(*)=O)c2)CC1 ; *CCC(=O)N1CCN(c2cccc(C(*)=O)c2)CC1 ; *CCCC(=O)N1CCN(c2cccc(C(*)=O)c2)CC1 ; *CCCCC(=O)N1CCN(c2cccc(C(*)=O)c2)CC1 ; *CCCCCC(=O)N1CCN(c2cccc(C(*)=O)c2)CC1 ; *CCCCCCC(=O)N1CCN(c2cccc(C(*)=O)c2)CC1]

In some aspects of Formula I, L is selected from:

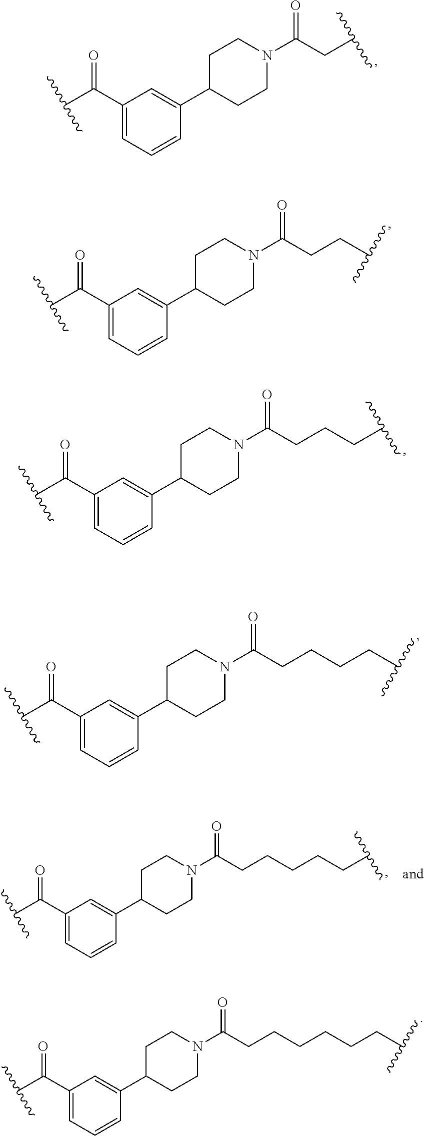 [SMILES: *CC(=O)N1CCC(c2cccc(C(*)=O)c2)CC1 ; *CCC(=O)N1CCC(c2cccc(C(*)=O)c2)CC1 ; *CCCC(=O)N1CCC(c2cccc(C(*)=O)c2)CC1 ; *CCCCC(=O)N1CCC(c2cccc(C(*)=O)c2)CC1 ; *CCCCCC(=O)N1CCC(c2cccc(C(*)=O)c2)CC1 ; *CCCCCCC(=O)N1CCC(c2cccc(C(*)=O)c2)CC1]

In some aspects of Formula I, L is selected from:

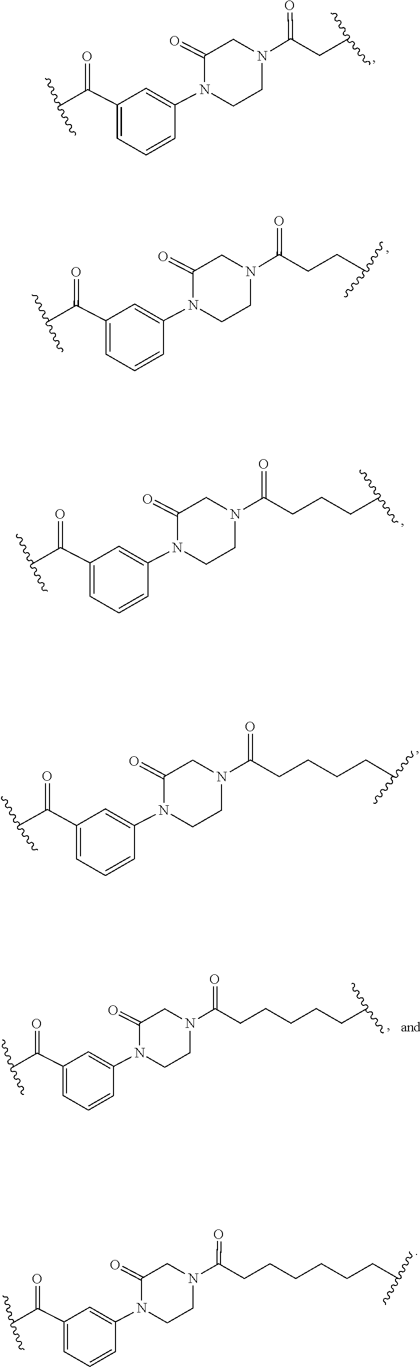 [SMILES: *CC(=O)N1CCN(c2cccc(C(*)=O)c2)C(=O)C1 ; *CCC(=O)N1CCN(c2cccc(C(*)=O)c2)C(=O)C1 ; *CCCC(=O)N1CCN(c2cccc(C(*)=O)c2)C(=O)C1 ; *CCCCC(=O)N1CCN(c2cccc(C(*)=O)c2)C(=O)C1 ; *CCCCCC(=O)N1CCN(c2cccc(C(*)=O)c2)C(=O)C1 ; *CCCCCCC(=O)N1CCN(c2cccc(C(*)=O)c2)C(=O)C1]

In some aspects of Formula I, L is selected from:

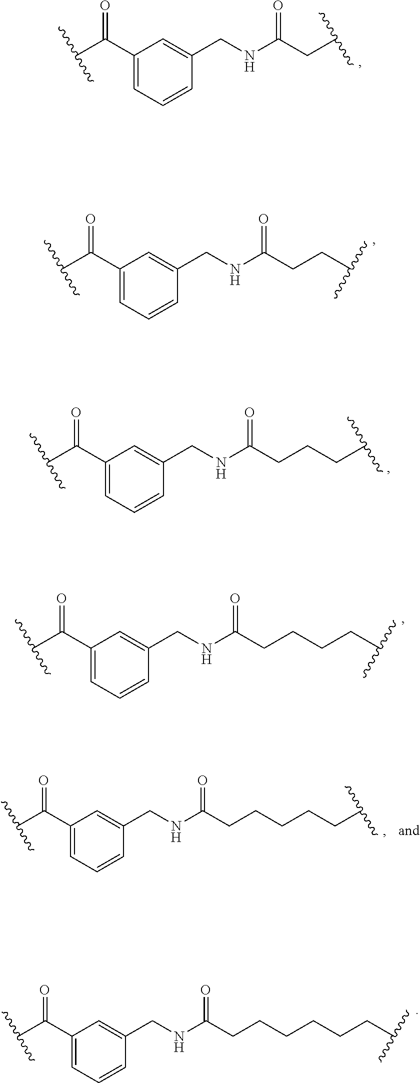 [SMILES: *CC(=O)NCc1cccc(C(*)=O)c1 ; *CCC(=O)NCc1cccc(C(*)=O)c1 ; *CCCC(=O)NCc1cccc(C(*)=O)c1 ; *CCCCC(=O)NCc1cccc(C(*)=O)c1 ; *CCCCCC(=O)NCc1cccc(C(*)=O)c1 ; *CCCCCCC(=O)NCc1cccc(C(*)=O)c1]

In some aspects of Formula I, L is selected from:

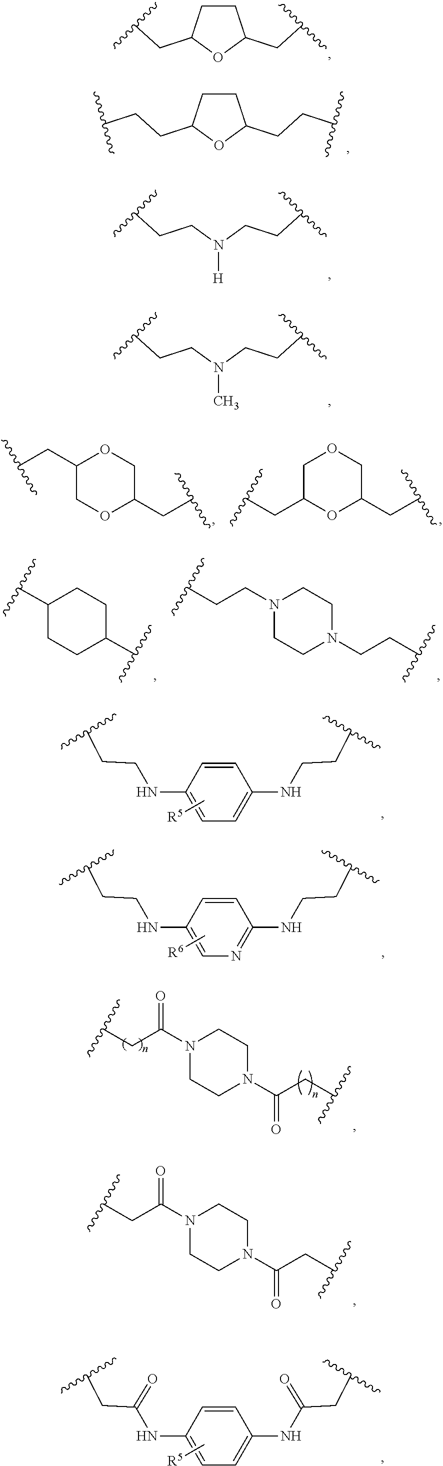

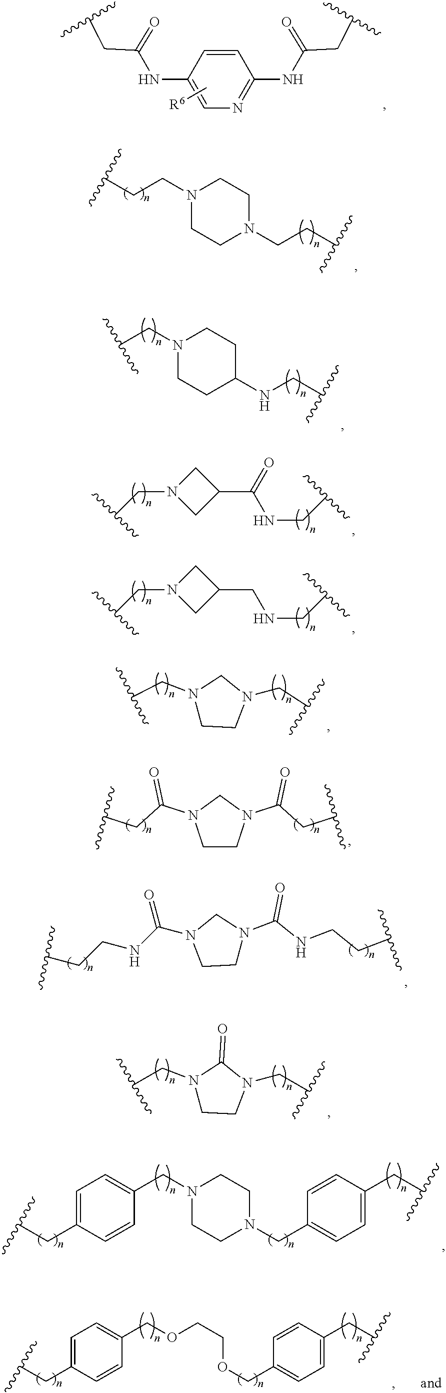

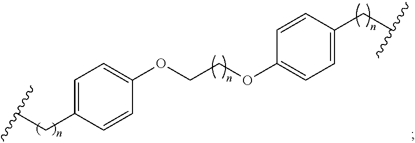

wherein n is independently selected at each occurrence from 1, 2, 3, 4, 5, and 6; and all other variables are as defined herein.

In some aspects of Formula I, X² is a bond. In some aspects of Formula I, X² is —NH—. In some aspects of Formula I, X² is —O—. In some aspects of Formula I, X² is —C≡C—. In some aspects of Formula I, X² is —C(═O)—. In some aspects of Formula I, X² is —S(═O)₂—. In some aspects of Formula I, X² is —C(═O) NH—. In some aspects of Formula I, X² is —NHC(═O)—. In some aspects of Formula I, X² is —S(═O)₂NH—. In some aspects of Formula I, X² is —NHS(═O)₂—. In some aspects of Formula I, X² is —NH(C═O)NH—.

The present disclosure also includes compounds of Formula I with at least one desired isotopic substitution of an atom at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one aspect, isotopically labeled compounds can be used in metabolic studies (with ¹⁴C), reaction kinetic studies (with, for example, ²H or ³H), detection or imaging techniques, such as positron emission tomography (PET), or single-photon emission computed tomography (SPECT) including drug and substrate tissue distribution assays, or in radioactive treatment of patients. In particular, an ¹⁸F0labeled compound may be particularly desirable for PET or SPECT studies. Isotopically labeled compounds of this disclosure and prodrugs thereof can generally be prepared by carrying out the procedures disclosed herein by substituting a readily available isotopically labeled reagent for a non-isotopically labeled reagent.

By way of a general example and without limitation, isotopes of hydrogen, such as deuterium (2H) and tritium (3H), may optionally be used anywhere in the described structures to achieve the desired result. Alternatively, or in addition, isotopes of carbon, e.g., ¹³C and ¹⁴C, may be used. In one aspect, the isotopic substitution is replacing hydrogen with deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, such as deuterium substitutions, can be partial or complete. Partial deuterium substitution means that at least one hydrogen is substituted with deuterium. In certain aspects, the isotope is 80, 85, 90, 95, or 99% or more enriched in an isotope at any location of interest. In some aspects, deuterium is 80, 85, 90, 95, or 99% enriched at a desired location. Unless otherwise stated, the enrichment at any point is above natural abundance and, in one aspect, is enough to alter a detectable property of the compounds as a drug in a human.

The compounds of the present disclosure may form a solvate with solvents (including water). Therefore, in one aspect, the disclosure includes a solvated form of the active compound. The term “solvate” refers to a molecular complex of a compound of the present disclosure (including a salt thereof) with one or more solvent molecules. Non-limiting examples of solvents are water, ethanol, dimethyl sulfoxide, acetone, and other common organic solvents. The term “hydrate” refers to a molecular complex comprising a disclosed compound and water. Pharmaceutically acceptable solvates in accordance with the disclosure include those wherein the solvent of crystallization may be isotopically substituted, e.g., D₂O, d₆-acetone, or d₆-DMSO. A solvate can be in a liquid or solid form.

A “prodrug.” as used herein, means a compound, which when administered to a host in vivo, is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain aspects, the prodrug renders the parent compound more lipophilic. In certain aspects, a prodrug can be provided that has several prodrug moieties in a linear, branched, or cyclic manner. For example, non-limiting aspects include the use of a divalent linker moiety such as a dicarboxylic acid, amino acid, diamine, hydroxycarboxylic acid, hydroxyamine, di-hydroxy compound, or another compound that has at least two functional groups that can link the parent compound with another prodrug moiety and is typically biodegradable in vivo. In some aspects, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some aspects, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched, or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort, may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion), and/or various types of wetting agents. As used herein, the term “carrier” encompasses, but is not limited to, any excipient, diluent, filler, salt, buffer, stabilizer, solubilizer, lipid, stabilizer, or other material well-known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants, and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include but are not limited to, any non-toxic, inert solid, semisolid or liquid filler, diluent, encapsulating material, or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain aspects, the preservative is an anti-oxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy (Polyethylene glycol)-1000], 1,2-Distearoyl-sn-glycero-3-Phosphoethanolamine-N-[Methoxy (Polyethylene glycol)-2000], and 1,2-Distearoyl-sn-glycero-3-Phosphoethanolamine-N-[Methoxy (Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain aspects, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, such as injectable aqueous or oleaginous suspensions, may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P., and isotonic sodium chloride solution. In addition, sterile, fixed oils are conventionally employed as a solvent or suspending medium. Any bland fixed oil can be employed, including synthetic mono- or diglycerides. In addition, fatty acids such as oleic acid are used in the preparation of injectables. In certain aspects, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter or by incorporating sterilizing agents in the form of sterile solid compositions, which can be dissolved or dispersed in sterile water or other sterile injectable media prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate-controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors, including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some aspects, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors, including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity and in vivo activity in animal models. Methods for the extrapolation of effective dosages in mice and other animals to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex, and extent of the disease in the patient and can be determined by one of skill in the art. The dosage can be adjusted by the individual physician in the event of any counterindications. Dosage can vary and can be administered in one or more doses daily for one or several days.

### Methods of Use

The present disclosure also provides methods for treating or preventing cancer in a subject, comprising administering to the subject a therapeutically effective amount of a compound or composition disclosed herein. The methods can further comprise administering one or more additional therapeutic agents, such as anti-cancer agents or anti-inflammatory agents. Additionally, the method can further comprise administering a therapeutically effective amount of ionizing radiation to the subject.

Methods of killing a cancer or tumor cell are also provided, comprising contacting the cancer or tumor cell with an effective amount of a compound or composition as described herein. In some aspects, the compounds induce degradation of CMG helicase. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow, pig, horse, or other animals, having an oncological disorder. In some aspects, the subject can receive the therapeutic compositions prior to, during, or after surgical intervention to remove part or all of a tumor.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue, and most invade surrounding tissues, can metastasize to several sites, are likely to recur after attempted removal, and may cause the death of the patient unless adequately treated. As used herein, the term neoplasia is used to describe all cancerous disease states and embraces or encompasses the pathological process associated with malignant, hematogenous, ascitic, and solid tumors. The cancers that may be treated by the compounds or compositions disclosed herein may comprise carcinomas, sarcomas, lymphomas, leukemias, germ cell tumors, or blastomas.

Carcinomas which may be treated by the compounds or compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas that may be treated by the compounds or compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compounds or compositions of the present disclosure may be used in the treatment of a lymphoma. Lymphomas that may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compounds or compositions of the present disclosure may be used in the treatment of a leukemia. Representative examples of leukemias include but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compounds or compositions of the present disclosure may be used in the treatment of a germ cell tumor, for example, germinomatous (such as germinoma, dysgerminoma, and seminoma), non-germinomatous (such as embryonal carcinoma, endodermal sinus tumor, choriocarcinoma, teratoma, polyembryoma, and gonadoblastoma) and mixed tumors.

The compounds compositions of the present disclosure may be used in the treatment of blastomas, for example, hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell syndrome, parathyroid cancer, carcinoma, multiple endocrine neoplasia phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDS-related cancers such as Kaposi sarcoma; epithelioid hemangioendothelioma; desmoplastic small round cell tumor; and liposarcoma.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier, such as an inert diluent or an assimilable edible carrier for oral delivery. In addition, the active compound can be incorporated into sustained-release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab. These other substances or radiation treatments can be given at the same time as or at different times from the compounds disclosed herein. Examples of other suitable chemotherapeutic agents include but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

In another aspect, methods are provided for the treatment of medical disorders associated with a helicase, for example, an SF3 and/or SF6 helicase, by administering a compound of Formula I, or pharmaceutically acceptable salts thereof. In particular aspects, the compounds described herein may be used in the treatment of cancer, either alone or in combination with one or more additional therapeutic agents, for example, a chemotherapeutic agent. In some aspects, the helicase comprises CMG helicase. In other aspects, the helicase comprises HPV E1 helicase.

Thus in one aspect, a method is provided for treating a cancer in a subject in need thereof, the method comprising administering a therapeutically effective amount of a compound of Formula I, or a pharmaceutically acceptable salt thereof. In some aspects, the compound of Formula I, or a pharmaceutically acceptable salt thereof, is administered as a pharmaceutical composition as further described herein. In some aspects, the subject is a human. In some aspects, the cancer is associated with dysregulation of a helicase, for example, an SF3 and/or SF6 helicase. In some aspects, the cancer is associated with CMG helicase. In some aspects, the cancer is associated with HPV E1 helicase.

In another aspect, a method is provided for treating cancers associated with elevated expression levels of Myc and/or elevated expression levels of Cyclin E. Elevated levels of Myc and Cyclin E have been associated overactivation of CMG helicases, leading to diminished reserve MCMs available to allow the cancer cell to successfully complete the S-phase of the cell cycle. Upon exposure of the cancer cell to a CMG helicase inhibitor such as those described herein, the cancer cell faces diminished survival and potentially cell death.

Thus, in one aspect, a method is provided for treating a cancer in a subject in need thereof, the method comprising:
(a) determining whether the cancer is characterized by elevated Myc expression and/or elevated Cyclin E expression; and
(b) if the cancer is determined to be characterized by elevated Myc expression and/or elevated Cyclin E expression in (a), administering a therapeutically effective amount of a compound of Formula I, or a pharmaceutically acceptable salt thereof, either alone or in combination with one or more additional therapeutic agents (such as a chemotherapeutic or cytotoxic agent).

In another aspect, a method of treating a cancer associated with elevated Myc expression and/or elevated Cyclin E expression is provided comprising administering a therapeutically effective amount of a compound of Formula I, or a pharmaceutically acceptable salt thereof, either alone or in combination with one or more additional therapeutic agents (such as a chemotherapeutic or cytotoxic agent).

In yet another aspect, a method is provided for inhibiting CMG helicase in a eukaryotic cell comprising contacting the cell with an effective amount of a compound of Formula I, or a pharmaceutically acceptable salt thereof, as described herein. In some aspects, the eukaryotic cell is a human cell.

In yet another aspect, a method for treating cancer in a subject in need thereof is provided, the method comprising:
(a) determining whether the cancer is associated with one or more signs of replicative stress; and
(b) if the cancer is determined to be associated with one or more signs of replicative stress, administering a therapeutically effective amount of a compound of Formula I, or a pharmaceutically acceptable salt thereof.

In another aspect, a method is provided for treating cancer in a subject in need thereof, wherein the cancer has been previously determined to be associated with one or more signs of replicative stress, the method comprising administering a therapeutically effective amount of a compound of Formula I, or a pharmaceutically acceptable salt thereof.

In some aspects, the one or more signs of replicative stress may comprise Myc overexpression, CyclinE overexpression, Rb loss, p53 loss, PolQ overexpression, or combinations thereof. In some aspects, the one or more signs or replicative stress comprise Myc overexpression. In some aspects, the one or more signs or replicative stress comprise CyclinE overexpression. In some aspects, the one or more signs or replicative stress comprise Rb loss. In some aspects, the one or more signs or replicative stress comprise p53 loss. In some aspects, the one or more signs or replicative stress comprise PolQ overexpression.

In yet another aspect, a method for treating cancer in a subject in need thereof is provided, the method comprising:
(a) determining whether the cancer harbors one or more inherited or acquired germ-line mutations; and
(b) if the cancer is determined to harbor one or more inherited or acquired germ-line mutations in (a), administering a therapeutically effective amount of a compound of Formula I, or a pharmaceutically acceptable salt thereof.

In another aspect, a method for treating cancer in a subject in need thereof is provided, wherein the cancer has been previously determined to harbor one or more inherited or acquired germ-line mutations, the method comprising administering a therapeutically effective amount of a compound of Formula I, or a pharmaceutically acceptable salt thereof.

In some aspects, the one or more inherited or acquired germ-line mutations may comprise loss of: p53, Rb, BRCA1, BRCA2, ATM, a xeroderma pigmentosum gene (such as XPA, XPB, XPC, XPD, XPE, XPF, or XPG), a mismatch repair gene (such as MSH2, MLH1, MSH6, PMS2), WRN, BLM, a Fanconi anemia gene (such as FANCA, FANCB, FANCC, FANCD2, FANCE, FANCF, FANCG, FANCI, FANCJ, FANCL, FANCM, FANCN, FANCO, FANCP, FANCQ, FANCT, FANCU, FANCV, or FANCW), NBS, Chek2, RecqL4, MYH, PALB2, BACH1, RAC51C, or combinations thereof.

In some aspects, the one or more inherited or acquired germ-line mutations comprise loss of p53. In some aspects, the one or more inherited or acquired germ-line mutations comprise loss of Rb. In some aspects, the one or more inherited or acquired germ-line mutations comprise loss of BRCA1. In some aspects, the one or more inherited or acquired germ-line mutations comprise loss of BRCA2. In some aspects, the one or more inherited or acquired germ-line mutations comprise loss of ATM. In some aspects, the one or more inherited or acquired germ-line mutations comprise loss of XPA. In some aspects, the one or more inherited or acquired germ-line mutations comprise loss of XPB. In some aspects, the one or more inherited or acquired germ-line mutations comprise loss of XPC. In some aspects, the one or more inherited or acquired germ-line mutations comprise loss of XPD. In some aspects, the one or more inherited or acquired germ-line mutations comprise loss of XPE. In some aspects, the one or more inherited or acquired germ-line mutations comprise loss of XPF. In some aspects, the one or more inherited or acquired germ-line mutations comprise loss of XPG. In some aspects, the one or more inherited or acquired germ-line mutations comprise loss of MSH2. In some aspects, the one or more inherited or acquired germ-line mutations comprise loss of MLH1. In some aspects, the one or more inherited or acquired germ-line mutations comprise loss of MSH6. In some aspects, the one or more inherited or acquired germ-line mutations comprise loss of PMS2. In some aspects, the one or more inherited or acquired germ-line mutations comprise loss of WRN. In some aspects, the one or more inherited or acquired germ-line mutations comprise loss of BLM. In some aspects, the one or more inherited or acquired germ-line mutations comprise loss of FANCA. In some aspects, the one or more inherited or acquired germ-line mutations comprise loss of FANCB. In some aspects, the one or more inherited or acquired germ-line mutations comprise loss of FANCC. In some aspects, the one or more inherited or acquired germ-line mutations comprise loss of FANCD2. In some aspects, the one or more inherited or acquired germ-line mutations comprise loss of FANCE. In some aspects, the one or more inherited or acquired germ-line mutations comprise loss of FANCF. In some aspects, the one or more inherited or acquired germ-line mutations comprise loss of FANCG. In some aspects, the one or more inherited or acquired germ-line mutations comprise loss of FANCI.

In some aspects, the one or more inherited or acquired germ-line mutations comprise loss of FANCJ. In some aspects, the one or more inherited or acquired germ-line mutations comprise loss of FANCL. In some aspects, the one or more inherited or acquired germ-line mutations comprise loss of FANCM. In some aspects, the one or more inherited or acquired germ-line mutations comprise loss of FANCN. In some aspects, the one or more inherited or acquired germ-line mutations comprise loss of FANCO. In some aspects, the one or more inherited or acquired germ-line mutations comprise loss of FANCP. In some aspects, the one or more inherited or acquired germ-line mutations comprise loss of FANCQ. In some aspects, the one or more inherited or acquired germ-line mutations comprise loss of FANCT. In some aspects, the one or more inherited or acquired germ-line mutations comprise loss of FANCU. In some aspects, the one or more inherited or acquired germ-line mutations comprise loss of FANCV. In some aspects, the one or more inherited or acquired germ-line mutations comprise loss of FANCW. In some aspects, the one or more inherited or acquired germ-line mutations comprise loss of NBS. In some aspects, the one or more inherited or acquired germ-line mutations comprise loss of Chek2. In some aspects, the one or more inherited or acquired germ-line mutations comprise loss of RecqL4. In some aspects, the one or more inherited or acquired germ-line mutations comprise loss of MYH. In some aspects, the one or more inherited or acquired germ-line mutations comprise loss of PALB2. In some aspects, the one or more inherited or acquired germ-line mutations comprise loss of BACH1. In some aspects, the one or more inherited or acquired germ-line mutations comprise loss of RAC51C.

In another aspect, a method is provided for treating an infection resulting from a papillomavirus in a subject in need thereof comprising administering a therapeutically effective amount of a compound described herein, or a pharmaceutically acceptable salt thereof, to the subject.

In some aspects, the papillomavirus is human papillomavirus (HPV). In some aspects, the HPV is an HPV strain selected from a strain including, but not limited to, HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, HPV82, or any other HPV strain which is known to result in an infection associated with a medical disorder.

In another aspect, a method is provided for treating a medical disorder associated with infection with human papillomavirus comprising administering to a subject in need thereof a therapeutically effective amount of a compound described herein or a pharmaceutically acceptable salt thereof. In some aspects, the medical disorder associated infection with human papillomavirus is cancer. Representative examples of medical disorders resulting from infection with HPV include, but are not limited to, common warts (associated with HPV2, HPV7, and HPV22, for example), plantar warts (associated with HPV1, HPV2, HPV4, and HPV63, for example), flat warts (associated with HPV3, HPV10, and HPV28, for example), anogenital warts (associated with HPV6, HPV11, HPV42, and HPV42, for example), genital cancers (associated with HPV16, HPV18, HPV26 HPV31, HPV33, HPV35, HPV39, HPV45, HPV51, HPV52, HPV53, HPV56, HPV58, HPV59, HPV66, HPV72, and HPV82, for example), epidermodysplasia verruciformis, focal epithelial hyperplasia (associated with HPV13 and HPV32, for example), mouth papillomas (associated with HPV6, HPV7, HPV11, HPV16, and HPV32, for example), oropharyngeal cancer (associated with HPV16, for example), verrucous cyst (associated with HPV60, for example), and laryngeal papillomatosis (associated with HPV6 and HPV11, for example).

In some aspects, the one or more additional therapeutic agents may comprise a Chk1 inhibitor, an ATR inhibitor, a Cdc7 inhibitor, or a Parp inhibitor.

In another aspect, a method for treating cancer in a subject in need thereof is provided, the method comprising:
(a) administering a therapeutically effective amount of a compound of Formula I, or a pharmaceutically acceptable salt thereof; and
(b) administering one or more additional therapeutic agents selected from a Chk1 inhibitor, an ATR inhibitor, a Cdc7 inhibitor, and a Parp inhibitor.

Representative Chk1 inhibitors which may be used in the above methods include, but are not limited to, AZD7762, Rabusertib (LY2603618), MK-8776 (SCH 900776), CHIR-124, PF-477736, prexasertib (LY2606368), GDC-0575, SAR-020106, CCT245737, and PD166285.

Representative ATR inhibitors which may be used in the above methods include, but are not limited to, VE-821, Berzosertib (VE-822), elimusertib (BAY-1895344), ETP-46464, CGK 733, AZ20, AZ31, ceralasertib (AZD6738), and VX-803 (M4344).

Representative examples of Cdc7 inhibitors which may be used in the above methods include, but are not limited to, XL-413, PHA-767491 (CAY10572), and LY3143921.

Representative examples of Parp inhibitors which may be used in the above methods include, but are not limited to, Olaparib, rucaparib, niraparib, talazoparib, veliparib, pamiparib (BGB-290), CEP 9722, E7016, 3-aminobenzamide,fluzoparib, AG-14361, A-966492, PJ34, UPF 1069, AZD2461, ME0328, BYK204165, BGP-15, RBN-2397, NU1025, E7449, 4-hydroxyquinazoline, NMS-P118, RBN012759, and picolinamide.

In view of the described compounds, compositions, and methods, hereinbelow are described certain more particular aspects of the disclosure. These particularly recited aspects should not, however, be interpreted to have any limiting effect on any different claims containing different or more general teachings described herein or that the “particular” aspects are somehow limited in some way other than the inherent meanings of the language and formulae literally used therein.

Aspect 1. A compound of Formula I

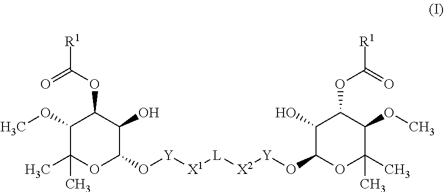

or a pharmaceutically acceptable salt thereof;
wherein:
R¹ is independently selected at each occurrence from monocyclic or bicyclic heteroaryl and monocyclic or bicyclic heterocycloalkyl, wherein each R¹ optionally includes at least one ring nitrogen atom substituted with R⁷ as allowed by valency, and wherein each R¹ is optionally substituted with 1, 2, 3, or 4 groups selected from R⁸ as allowed by valency;
Y is independently selected at each occurrence from a bond, bicyclic aryl, or bicyclic heteroaryl, wherein each Y is optionally substituted with 1, 2, 3, or 4 groups independently selected from R² as allowed by valency;
X¹ is selected from a bond or —NR^{a}—;
L is selected from a bond or L¹-L²-L³-L⁴-L⁵-L⁶-L⁷-;
L¹, L², L³, L⁴, L⁵, L⁶, and L⁷ are independently selected from:
a) a bond;

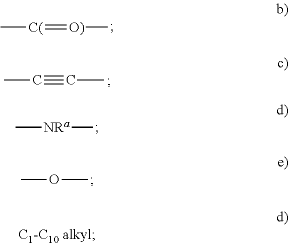

e) cycloalkyl optionally substituted with 1, 2, 3, or 4 substituents independently selected from R³;
f) heterocycloalkyl optionally substituted with 1, 2, 3, or 4 substituents independently selected from R⁴;
g) aryl optionally substituted with 1, 2, 3, or 4 groups independently selected from R⁵;
h) heteroaryl optionally substituted with 1, 2, 3, or 4 groups independently selected from R⁶;

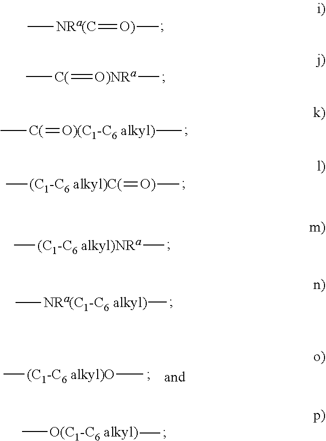

X² is selected from a bond, —NR^{a}—, —O—, —C≡C—, —C(═O)—, —S(═O)₂—, —C(═O) NR^{as}—, —NR^{a}C(═O)—, —S(═O)₂NR^{a}—, —NR^{a}S(═O)₂—, and —NR^{a}(C═O)NR^{b}—;
R² is selected at each occurrence from halogen, hydroxy, nitro, —NHR^{a}, C₁-C₆ alkyl, C₁-C₆ haloalkyl, aryl, and oxo;
R³ and R⁴ are independently selected at each occurrence from halogen, hydroxy, —NHR^{a}, C₁-C₆ alkyl, C₁-C₆ haloalkyl and oxo;
R⁵ and R⁶ are independently selected at each occurrence from halogen, hydroxy, nitro,
   —NHR^{a}, C₁-C₆ alkyl, C₁-C₆ haloalkyl, and aryl;
R⁷ is selected from: hydrogen; C₁-C₄ alkyl; C₂-C₆ alkenyl; C₂-C₆ alkynyl; C₃-C₆ cycloalkyl; C₁-C₆ alkoxy; and (C₀-C₆ alkyl) (aryl) which is optionally substituted with halogen, C₁-C₆ alkyl, or C₁-C₆ alkoxy;
R⁸ is selected from: hydrogen; halogen; hydroxy; —NR^{a}R^{b}; nitro; C₁-C₆ alkyl; C₁-C₆ haloalkyl; C₃-C₆ cycloalkyl; C₃-C₆ heterocycloalkyl; C₂-C₆ alkenyl; C₂-C₆ alkynyl; C₁-C₆ alkoxy; (C₀-C₆ alkyl) (aryl) which is optionally substituted with halogen, C₁-C₆ alkyl, or C₁-C₆ alkoxy; and (C₀-C₆ alkyl) (heteroaryl) which is optionally substituted with halogen, C₁-C₆ alkyl, or C₁-C₆ alkoxy; and
R^{a} and R^{b} are independently selected at each occurrence from hydrogen and C₁-C₆ alkyl.
   Aspect 2. The compound of aspect 1, wherein R¹ is

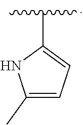 [SMILES: *c1ccc(C)[nH]1]

Aspect 3. The compound of aspect 1, wherein R¹ is selected from:

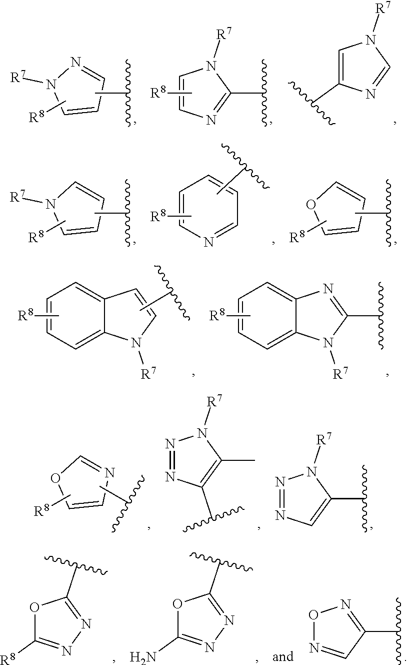

Aspect 4. The compound of any one of aspect 1, wherein R¹ is selected from:

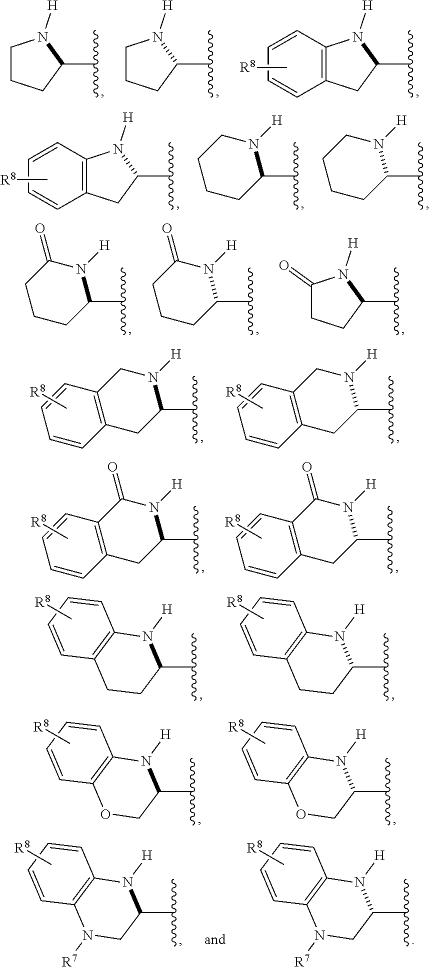

Aspect 5. The compound of any one of aspects 1-4, wherein Y is a bond.
Aspect 6. The compound of any one of aspects 1-4, wherein Y is

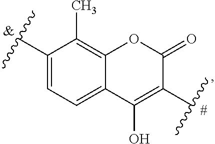 [SMILES: *c1ccc2c(O)c(*)c(=O)oc2c1C]

wherein & denotes the point of attachment to the neighboring oxygen atom and #denotes the point of attachment to X¹ and/or X².
Aspect 7. The compound of any one of aspects 1-4, wherein Y is selected from:

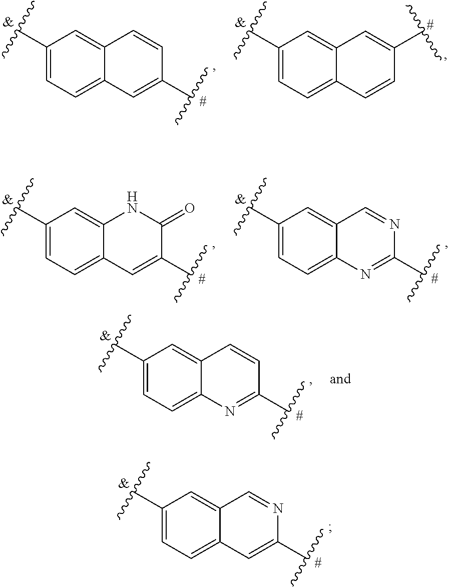 [SMILES: *c1ccc2cc(*)ccc2c1 ; *c1ccc2ccc(*)cc2c1 ; *c1ccc2cc(*)c(=O)[nH]c2c1 ; *c1ccc2nc(*)ncc2c1 ; *c1ccc2nc(*)ccc2c1 ; *c1ccc2cc(*)ncc2c1]

wherein & denotes the point of attachment to the neighboring oxygen atom and #denotes the point of attachment to X¹ and/or X².
Aspect 8. The compound of any one of aspects 1-7, wherein X¹ is —NH—.
Aspect 9. The compound of any one of aspects 1-8, wherein L¹, L², L³, L⁴, L⁵, L⁶, and L⁷ are each independently selected from:
a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O)NH—; —C(═O)CH₂—; —C(═O)(CH₂)₂—; —C(═O)(CH₂)₃—; —C(═O)(CH₂)₄—; —C(═O)(CH₂)₅—; —C(═O)(CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)₅—; —O(CH₂)₆—;

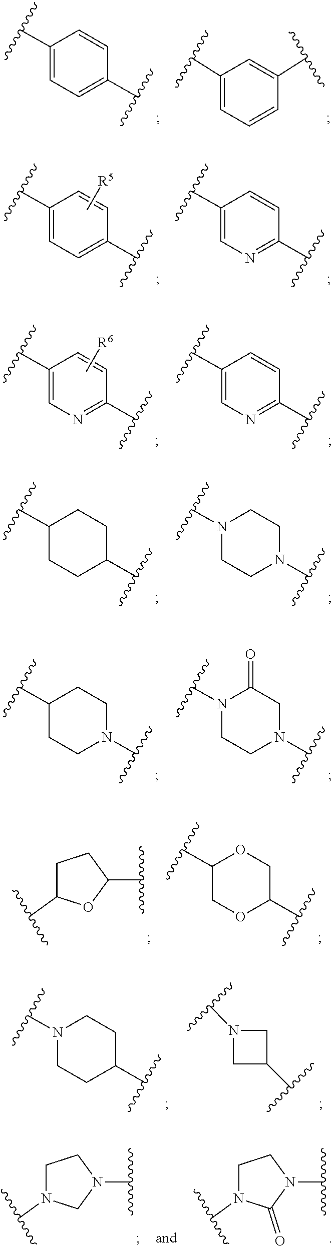

wherein L¹, L², L³, L⁴, L⁵, L⁶, and L⁷ are selected in such a way that: no two —C(═O)— moieties are adjected to each other; no two —O— or —NH— moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of L¹, L², L³, L⁴, L⁵, L⁶, and L⁷.
   Aspect 10. The compound of any one of aspects 1-8, wherein L is selected from:

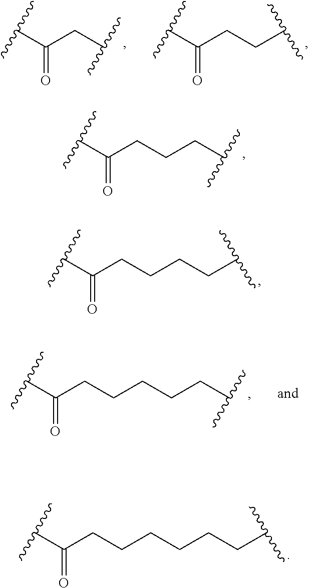 [SMILES: *CC(*)=O ; *CCC(*)=O ; *CCCC(*)=O ; *CCCCC(*)=O ; *CCCCCC(*)=O ; *CCCCCCC(*)=O]

Aspect 11. The compound of any one of aspects 1-8, wherein L is selected from:

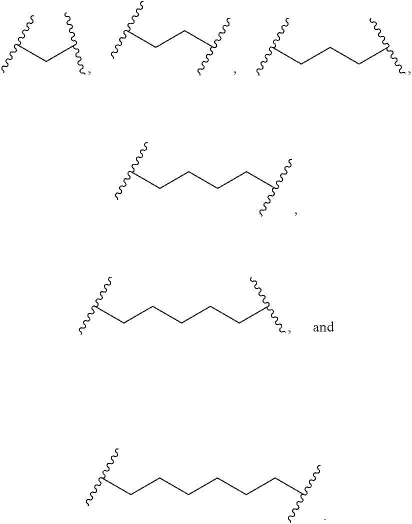

Aspect 12. The compound of any one of aspects 1-8, wherein L is selected from:

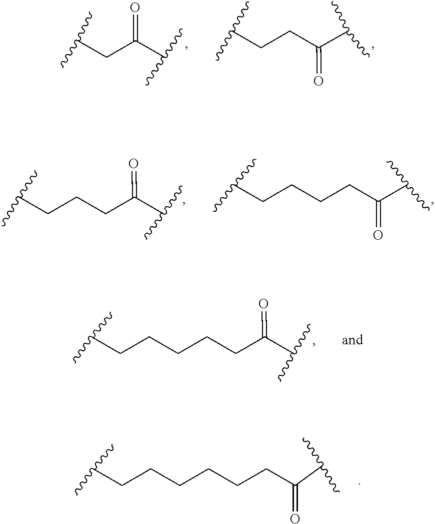 [SMILES: *CC(*)=O ; *CCC(*)=O ; *CCCC(*)=O ; *CCCCC(*)=O ; *CCCCCC(*)=O ; *CCCCCCC(*)=O]

Aspect 13. The compound of any one of aspects 1-8, wherein L is

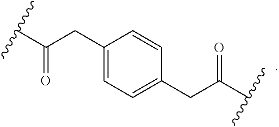 [SMILES: *C(=O)Cc1ccc(CC(*)=O)cc1]

Aspect 14. The compound of any one of aspects 1-8, wherein L is

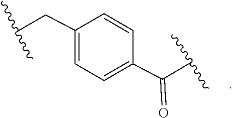 [SMILES: *Cc1ccc(C(*)=O)cc1]

Aspect 15. The compound of any one of aspects 1-8, wherein L is selected from:

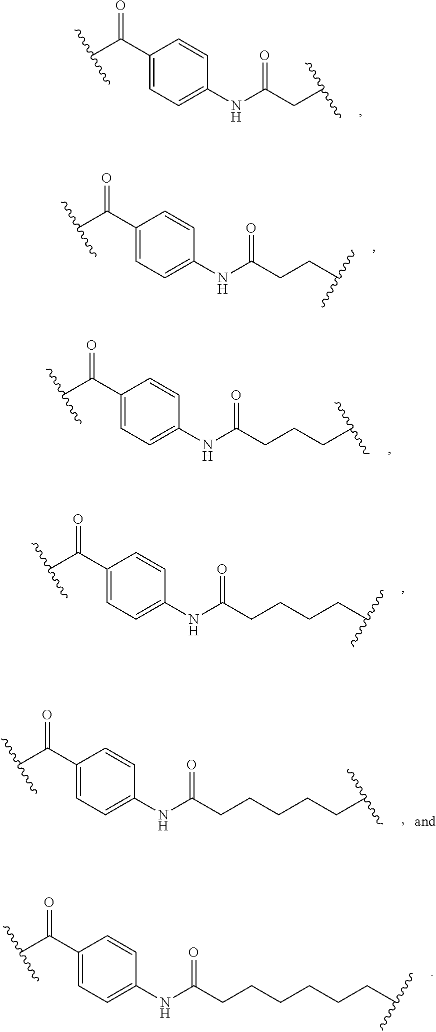 [SMILES: *CC(=O)Nc1ccc(C(*)=O)cc1 ; *CCC(=O)Nc1ccc(C(*)=O)cc1 ; *CCCC(=O)Nc1ccc(C(*)=O)cc1 ; *CCCCC(=O)Nc1ccc(C(*)=O)cc1 ; *CCCCCC(=O)Nc1ccc(C(*)=O)cc1 ; *CCCCCCC(=O)Nc1ccc(C(*)=O)cc1]

Aspect 16. The compound of any one of aspects 1-8, wherein L is selected from:

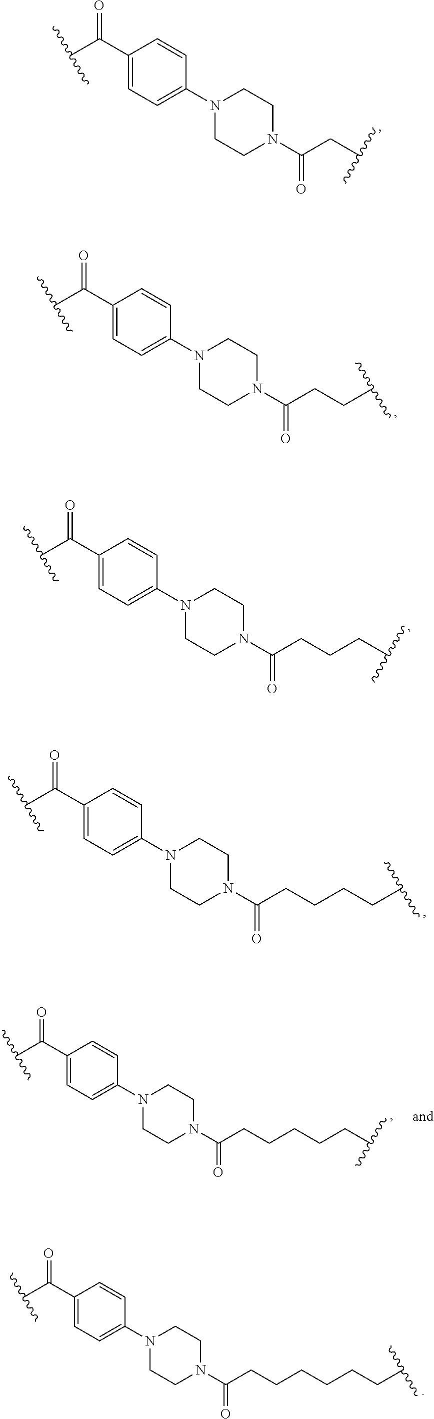 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

Aspect 17. The compound of any one of aspects 1-8, wherein L is selected from:

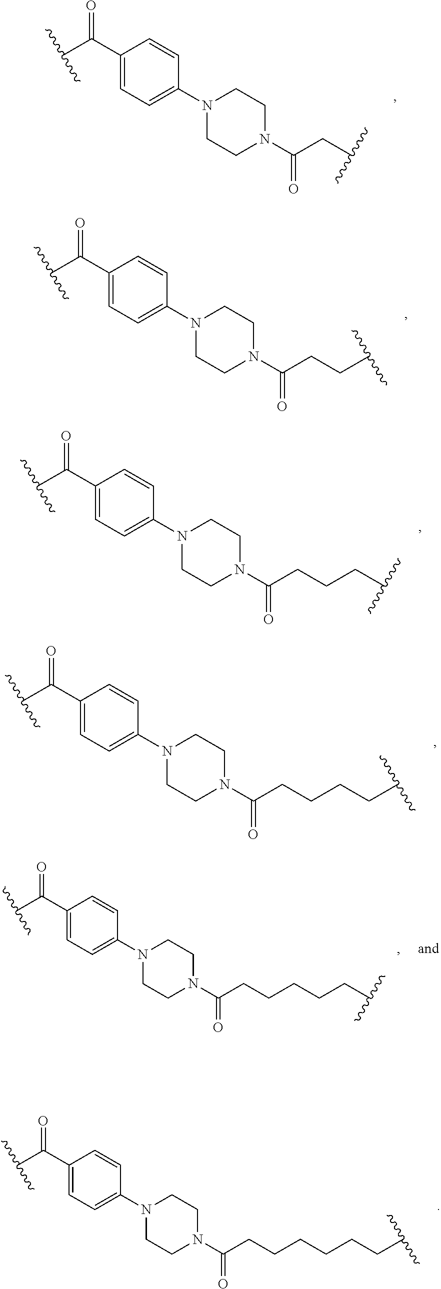 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

Aspect 18. The compound of any one of aspects 1-8, wherein L is selected from:

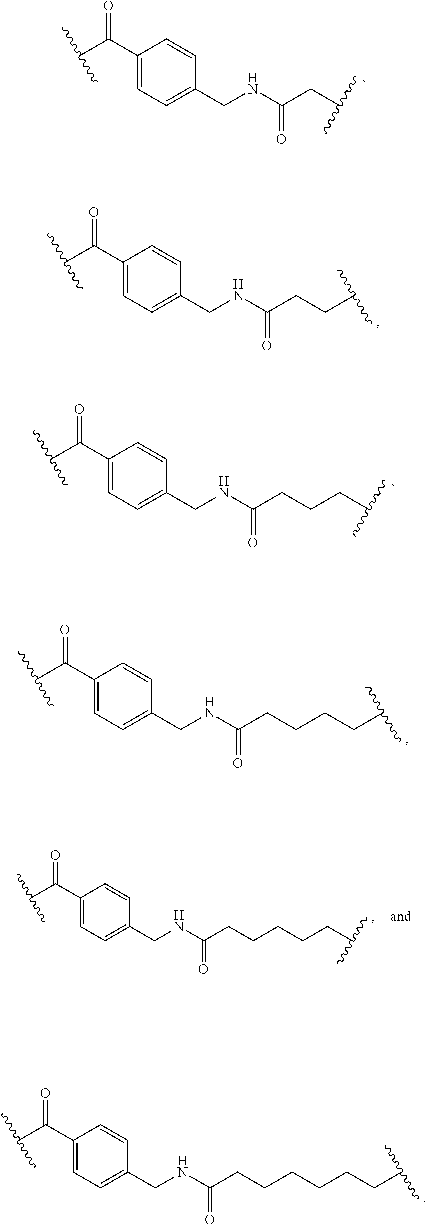 [SMILES: *CC(=O)NCc1ccc(C(*)=O)cc1 ; *CCC(=O)NCc1ccc(C(*)=O)cc1 ; *CCCC(=O)NCc1ccc(C(*)=O)cc1 ; *CCCCC(=O)NCc1ccc(C(*)=O)cc1 ; *CCCCCC(=O)NCc1ccc(C(*)=O)cc1 ; *CCCCCCC(=O)NCc1ccc(C(*)=O)cc1]

Aspect 19. The compound of any one of aspects 1-8, wherein L is selected from:

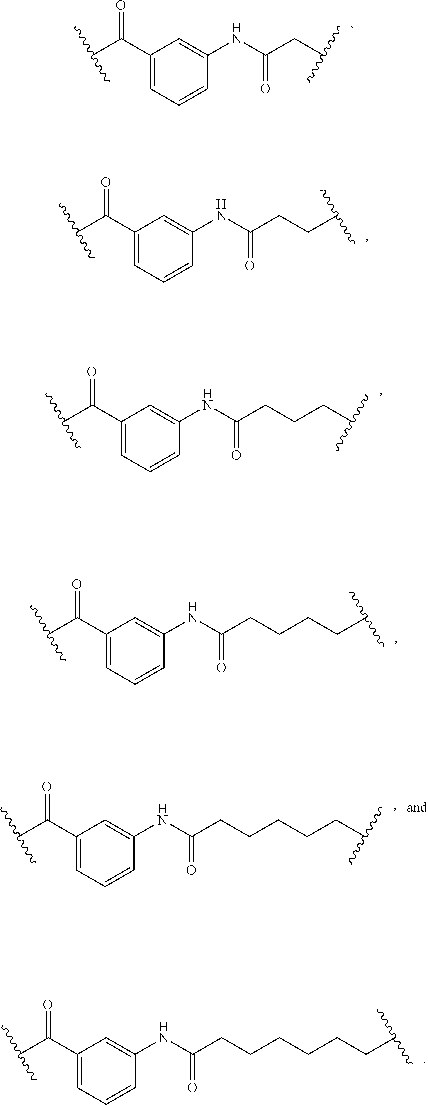 [SMILES: *CC(=O)Nc1cccc(C(*)=O)c1 ; *CCC(=O)Nc1cccc(C(*)=O)c1 ; *CCCC(=O)Nc1cccc(C(*)=O)c1 ; *CCCCC(=O)Nc1cccc(C(*)=O)c1 ; *CCCCCC(=O)Nc1cccc(C(*)=O)c1 ; *CCCCCCC(=O)Nc1cccc(C(*)=O)c1]

Aspect 20. The compound of any one of aspects 1-8, wherein L is selected from:

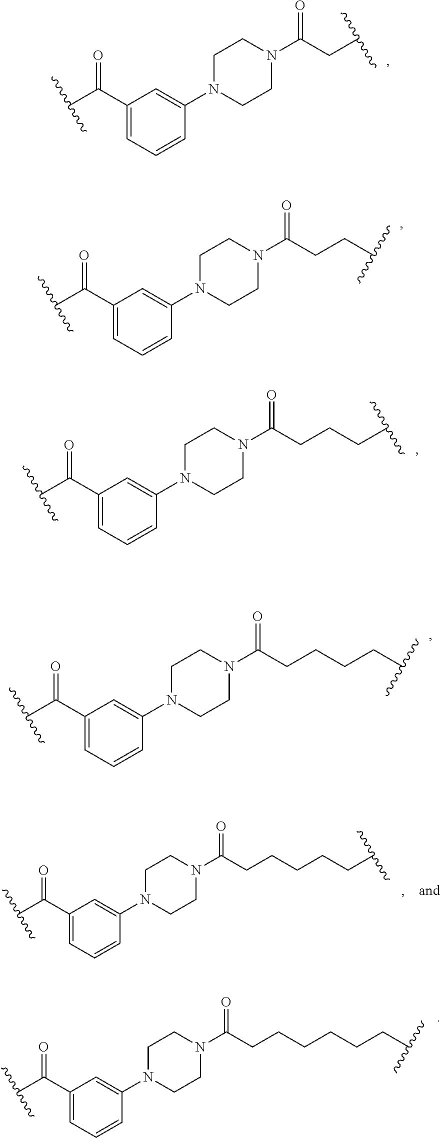 [SMILES: *CC(=O)N1CCN(c2cccc(C(*)=O)c2)CC1 ; *CCC(=O)N1CCN(c2cccc(C(*)=O)c2)CC1 ; *CCCC(=O)N1CCN(c2cccc(C(*)=O)c2)CC1 ; *CCCCC(=O)N1CCN(c2cccc(C(*)=O)c2)CC1 ; *CCCCCC(=O)N1CCN(c2cccc(C(*)=O)c2)CC1 ; *CCCCCCC(=O)N1CCN(c2cccc(C(*)=O)c2)CC1]

Aspect 21. The compound of any one of aspects 1-8, wherein L is selected from:

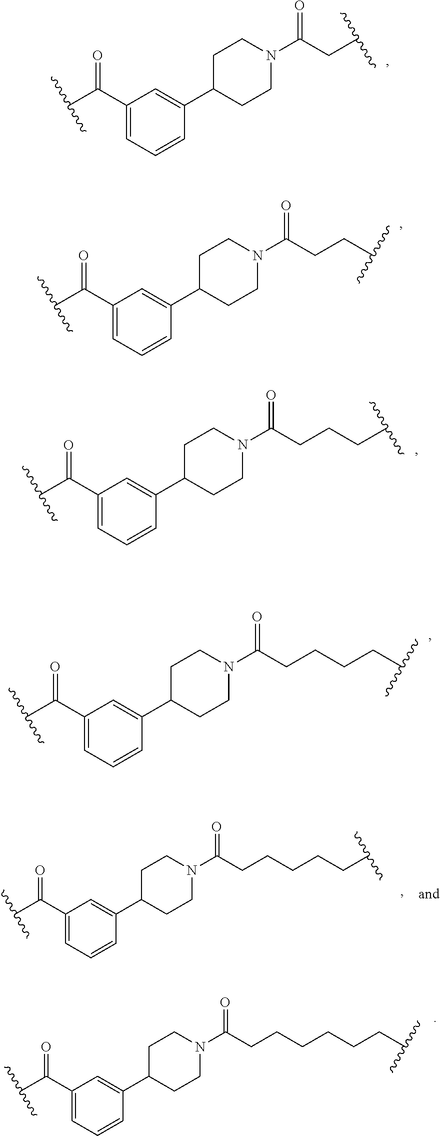 [SMILES: *CC(=O)N1CCC(c2cccc(C(*)=O)c2)CC1 ; *CCC(=O)N1CCC(c2cccc(C(*)=O)c2)CC1 ; *CCCC(=O)N1CCC(c2cccc(C(*)=O)c2)CC1 ; *CCCCC(=O)N1CCC(c2cccc(C(*)=O)c2)CC1 ; *CCCCCC(=O)N1CCC(c2cccc(C(*)=O)c2)CC1 ; *CCCCCCC(=O)N1CCC(c2cccc(C(*)=O)c2)CC1]

Aspect 22. The compound of any one of aspects 1-8, wherein L is selected from:

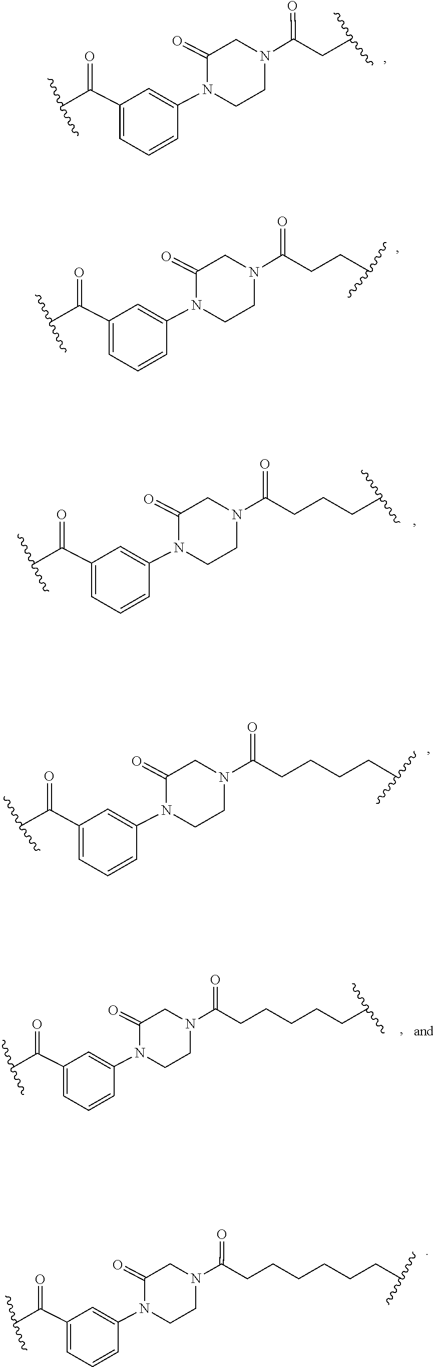 [SMILES: *CC(=O)N1CCN(c2cccc(C(*)=O)c2)C(=O)C1 ; *CCC(=O)N1CCN(c2cccc(C(*)=O)c2)C(=O)C1 ; *CCCC(=O)N1CCN(c2cccc(C(*)=O)c2)C(=O)C1 ; *CCCCC(=O)N1CCN(c2cccc(C(*)=O)c2)C(=O)C1 ; *CCCCCC(=O)N1CCN(c2cccc(C(*)=O)c2)C(=O)C1 ; *CCCCCCC(=O)N1CCN(c2cccc(C(*)=O)c2)C(=O)C1]

Aspect 23. The compound of any one of aspects 1-8, wherein L is selected from:

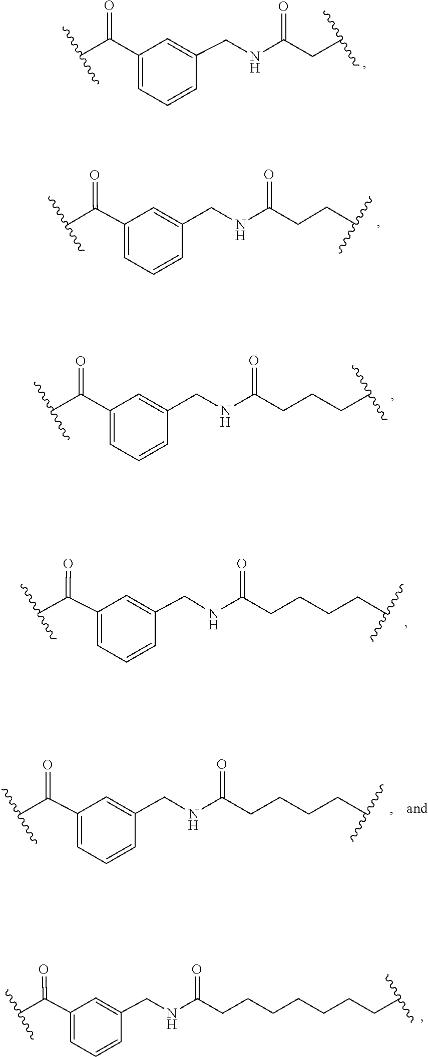 [SMILES: *CC(=O)NCc1cccc(C(*)=O)c1 ; *CCC(=O)NCc1cccc(C(*)=O)c1 ; *CCCC(=O)NCc1cccc(C(*)=O)c1 ; *CCCCC(=O)NCc1cccc(C(*)=O)c1 ; *CCCCC(=O)NCc1cccc(C(*)=O)c1 ; *CCCCCCCC(=O)NCc1cccc(C(*)=O)c1]

Aspect 24. The compound of any one of aspects 1-8, wherein L is selected from:

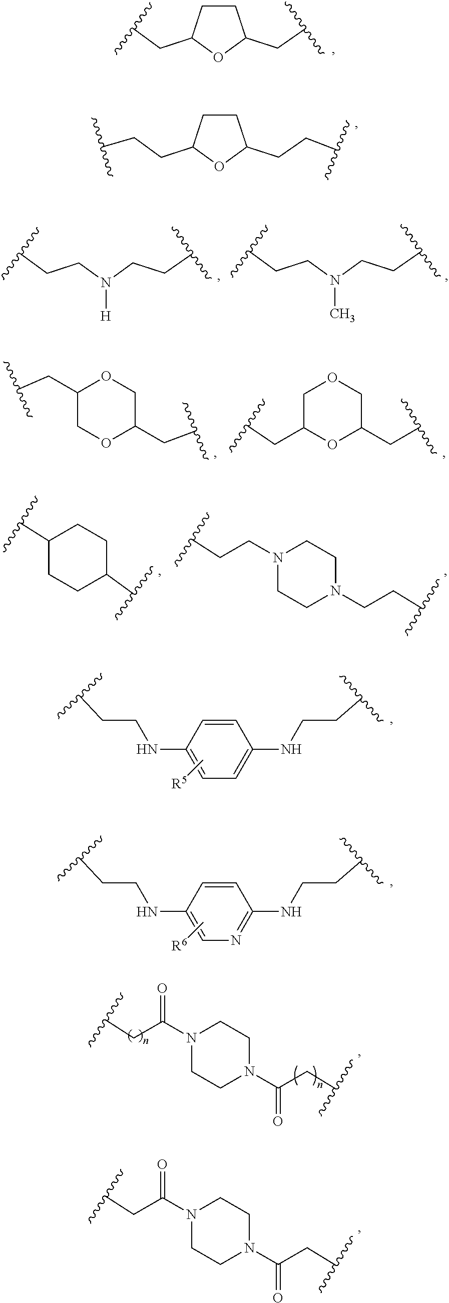

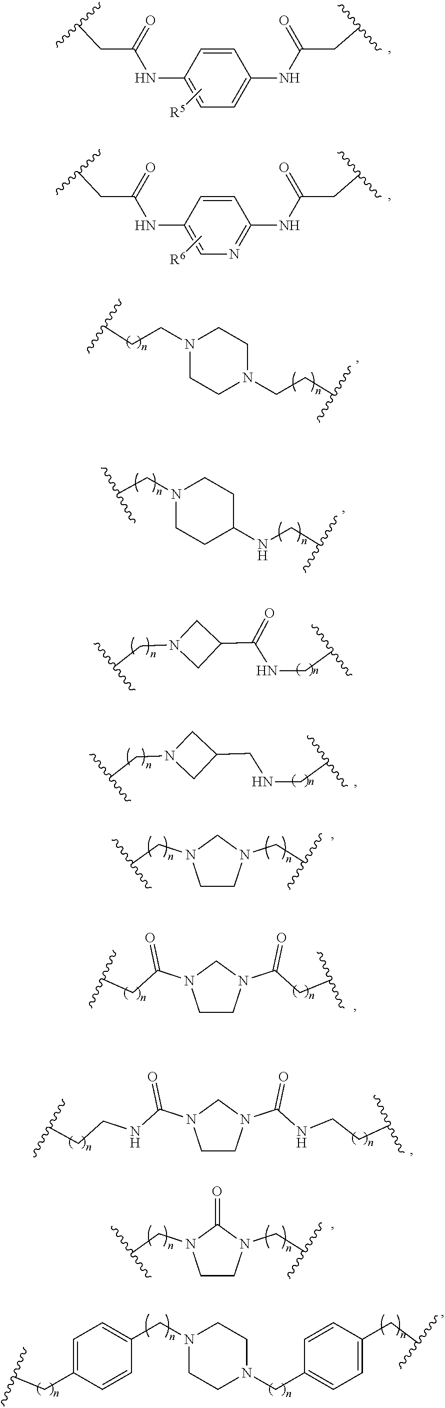

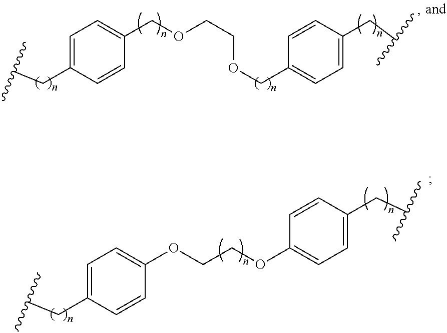

wherein n is independently selected at each occurrence from 1, 2, 3, 4, 5, and 6.
Aspect 25. The compound of any one of aspects 1-24, wherein X² is a bond.
Aspect 26. The compound of any one of aspects 1-24, wherein X² is —NH—.
Aspect 27. The compound of any one of aspects 1-24, wherein X² is —O—.
Aspect 28. The compound of any one of aspects 1-24, wherein X² is —C≡C—.
Aspect 29. The compound of any one of aspects 1-24, wherein X² is —C(═O)—.
Aspect 30. The compound of any one of aspects 1-24, wherein X² is —S(═O)₂—.
Aspect 31. The compound of any one of aspects 1-24, wherein X² is —C(═O)NH—.
Aspect 32. The compound of any one of aspects 1-24, wherein X² is —NHC(═O)—.
Aspect 33. The compound of any one of aspects 1-24, wherein X² is —S(═O)₂NH—.
Aspect 34. The compound of any one of aspects 1-24, wherein X² is —NHS(═O)₂—.
Aspect 35. The compound of any one of aspects 1-24, wherein X² is —NH(C═O)NH—.
Aspect 36. A pharmaceutical composition comprising a compound of any one of aspects 1-35, or a pharmaceutically acceptable salt thereof, and a pharmaceutically acceptable carrier or excipient.
Aspect 37. A method for treating cancer in a subject in need thereof comprising administering to the subject a therapeutically effective amount of a compound of any one of aspects 1-35, or a pharmaceutically acceptable salt thereof, or a pharmaceutical composition of aspect 36.
Aspect 38. The method of aspect 37, wherein the cancer is associated with or mediated by a helicase.
Aspect 39. The method of aspect 38, wherein the helicase is an SF3 helicase.
Aspect 40. The method of aspect 39, wherein the helicase is HPV E1 helicase.
Aspect 41. The method of aspect 38, wherein the helicase is an SF6 helicase.
Aspect 42. The method of aspect 41, wherein the helicase is CMG helicase.
Aspect 43. The method of any one of aspects 37 or 38, wherein the cancer is associated with overactivation of CMG helicase.
Aspect 44. The method of any one of aspects 37 or 38, wherein the cancer is associated with an infection by a papillomavirus.
Aspect 45. The method of aspect 44, wherein the papillomavirus is human papillomavirus (HPV).
Aspect 46. A method for treating cancer in a subject in need thereof comprising:
(a) determining whether the cancer is associated with elevated expression of Myc and/or elevated expression of Cyclin E; and
(b) if the cancer is determined to be associated with elevated expression of Myc and/or elevated expression of Cyclin E in (a), administering a therapeutically effective amount of a compound of any one of aspects 1-35, or a pharmaceutically acceptable salt thereof, or a pharmaceutical composition of aspect 36.
   Aspect 47. A method for treating cancer in a subject in need thereof, wherein the cancer has been previously determined to be associated with elevated expression of Myc and/or elevated expression of Cyclin E, the method comprising administering a therapeutically effective amount of a compound of any one of aspects 1-35, or a pharmaceutically acceptable salt thereof, or a pharmaceutical composition of aspect 36.
   Aspect 48. The method of any one of aspects 46 or 47, wherein the cancer is associated with overactivation of CMG helicase.
   Aspect 49. The method of any one of aspects 37-48, wherein the compound or pharmaceutical composition is administered in combination or alternation with one or more additional therapeutic agents.
   Aspect 50. The method of aspect 49, wherein the one or more additional therapeutic agents are a chemotherapeutic or cytotoxic agent.
   Aspect 51. A method of treating an infection with a papillomavirus in a subject in need thereof comprising administering a therapeutically effective amount of a compound of any one of aspects 1-35, or a pharmaceutically acceptable salt thereof, or a pharmaceutical composition of aspect 36.
   Aspect 52. The method of aspect 51, wherein the papillomavirus comprises human papillomavirus.
   Aspect 53. The method of aspect 52, wherein the human papillomavirus comprises a strain selected from HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, and HPV82.
   Aspect 54. The method of aspect 52, wherein the human papillomavirus comprises a strain selected from HPV16, HPV18, HPV31, and HPV45.
   Aspect 55. The method of any one of aspects 51-54, wherein the human papillomavirus is associated with a cancer.
   Aspect 56. The method of aspect 55, wherein the cancer is selected from cervical cancer, vulvar cancer, vaginal cancer, penile cancer, anal cancer, rectal cancer, oropharyngeal cancer, and head and neck cancer.
   Aspect 57. A method for inhibiting a helicase in a eukaryotic cell comprising contacting the cell with an effective amount of a compound of any one of aspects 1-35, or a pharmaceutically acceptable salt thereof.
   Aspect 58. The method of aspect 57, wherein the helicase is an SF3 helicase.
   Aspect 59. The method of any one of aspects 57 or 58, wherein the helicase is HPV E1 helicase.
   Aspect 60. The method of aspect 57, wherein the helicase is an SF6 helicase.
   Aspect 61. The method of any one of aspects 57 or 60, wherein the helicase is CMG helicase.
   Aspect 62. A method for inhibiting replication of a papillomavirus in a eukaryotic cell comprising contacting the cell with an effective amount of a compound of any one of aspects 1-35, or a pharmaceutically acceptable salt thereof.
   Aspect 63. The method of aspect 62, wherein the papillomavirus is human papillomavirus.
   Aspect 64. The method of aspect 63, wherein the human papillomavirus comprises a strain selected from HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, and HPV82.
   Aspect 65. The method of 63, wherein the human papillomavirus comprises a strain selected from HPV16, HPV18, HPV31, and HPV45.
   Aspect 66. The method of any one of aspects 57-65, wherein the eukaryotic cell is a human cell.
   Aspect 67. a method for treating cancer in a subject in need thereof comprising:
(a) determining whether the cancer harbors one or more inherited or acquired germ-line mutations; and
(b) if the cancer is determined to harbor one or more inherited or acquired germ-line mutations in (a), administering to the subject a therapeutically effective amount of a compound of any one of aspects 1-35, or a pharmaceutically acceptable salt thereof, or a pharmaceutical composition of aspect 36.
   Aspect 68. A method for treating cancer in a subject in need thereof, wherein the cancer has been previously determined to harbor one or more inherited or acquired germ-line mutations, the method comprising administering to the subject a therapeutically effective amount of a compound of any one of aspects 1-35, or a pharmaceutically acceptable salt thereof, or a pharmaceutical composition of aspect 36.
   Aspect 69. The method of aspect 67 or aspect 68, wherein the one or more inherited or acquired germ-line mutations comprises loss of: p53, Rb, BRCA1, BRCA2, ATM, a xeroderma pigmentosum gene (such as XPA, XPB, XPC, XPD, XPE, XPF, or XPG), a mismatch repair gene (such as MSH2, MLH1, MSH6, PMS2), WRN, BLM, a Fanconi anemia gene (such as FANCA, FANCB, FANC, FANCD2, FANCE, FANCF, FANCG, FANCI, FANCJ, FANCL, FANCM, FANCN, FANCO, FANCP, FANCQ, FANCT, FANCU, FANCV, or FANCW), NBS, Chek2, RecqL4, MYH, PALB2, BACH1, RAC51C, or combinations thereof.
   Aspect 70. The method of any one of aspects 67-69, wherein the compound is administered in combination with an additional therapeutic agent.
   Aspect 71. The method of aspect 70, wherein the additional therapeutic agent is selected from a Chk1 inhibitor, an ATR inhibitor, a Cdc7 inhibitor, or a Parp inhibitor.
   Aspect 72. A method for treating cancer in a subject in need thereof comprising:
(a) administering to the subject a therapeutically effective amount of a compound of any one of aspects 1-35, or a pharmaceutically acceptable salt thereof, or a pharmaceutical composition of aspect 36; and
(b) administering an additional therapeutic agent selected from a Chk1 inhibitor, an ATR inhibitor, a Cdc7 inhibitor, and a Parp inhibitor.

A number of aspects of the disclosure have been described. Nevertheless, it will be understood that various modifications may be made without departing from the spirit and scope of the disclosure. Accordingly, other aspects are within the scope of the following claims.

By way of non-limiting illustration, examples of certain aspects of the present disclosure are given below.

### Examples

The following examples are set forth below to illustrate the compounds, compositions, and methods claimed herein, along with associated methods and results according to the disclosed subject matter. These examples are not intended to be inclusive of all aspects of the subject matter disclosed herein, but rather to illustrate representative methods and results. These examples are not intended to exclude equivalents and variations of the present disclosure, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Inhibitors of CMG Helicase Using Coumermycin Analogs

The synthesis of coumermycin analogs 5 and 9 shown in Scheme 1 is carried out using known synthetic protocols from readily available Novobiocin (see Laurin P, Ferroud D, Klich M, Dupuis —Hamelin C, Mauvais P, Lassaigne P, Bonnefoy A, Musicki B. Synthesis and in vitro evaluation of novel highly potent coumarin inhibitors of gyrase B. Bioorganic & Medicinal Chemistry Letters. 1999; 9(14):2079-84; and WO01/87309). Under acidic reaction conditions in acetone, Novobiocin provides key intermediate 1. Hydrolysis of the carbamate group of 1 generates hydroxyacetonide intermediate 2. The pyrrole-anhydride building block 3 is synthesized from commercially available ethyl 5-methyl-1H-pyrrole-2-carboxylate via two steps; first hydrolysis of the ester to obtain the carboxylic acid and coupling the resulting acid via peptide coupling conditions (e.g. EDCI, DCM) to obtain the required anhydride 3 (see Olson SH, Slossberg LH. Synthesis of coumermycin A1. Tetrahedron Letters. 2003; 44 (1): 61-3). Acylation, catalyzed by tributylphosphine, of the hydroxyl group in the sugar fragment 2 with the pyrrole anhydride 3 provides pyrrole carbamate 4, which is subjected to acidic conditions to generate pyrrole-noviose 5. The coumermycin building block 6 shown in Scheme 2 is synthesized using known synthetic procedures (see Musicki B, Periers A-M, Laurin P, Ferroud D, Benedetti Y, Lachaud S, Chatreaux F, Haesslein J-L, Iltis A, Pierre C, Khider J, Tessot N, Airault M, Demassey J, Dupuis —Hamelin C, Lassaigne P, Bonnefoy A, Vicat P, Klich M. Improved antibacterial activities of coumarin antibiotics bearing 5′,5′-dialkylnoviose: biological activity of RU79115. Bioorganic & Medicinal Chemistry Letters. 2000; 10(15):1695-9) from readily available 2,4-dihydroxy-3-methylacetophenone 10, first by selectively protecting the 4-hydroxyl group with dihydropyran to generate the protected acetophenone 11 and further reaction with diethyl carbonate to provide the coumarin intermediate 12. The coumarin precursor 13 is generated from 12 by reacting with diphenylazodimethane and deprotection of the 4-hydroxyl group which generates required coumarin fragment 6. The coumarin intermediate 6 is used to generate compound 7 using Mitsunobu reaction conditions. The hydrogenation of compound 7 using Pd/C followed by diazotization of the resulting intermediate facilitates formation of key intermediate amino-coumarin 9 shown in Scheme 1.

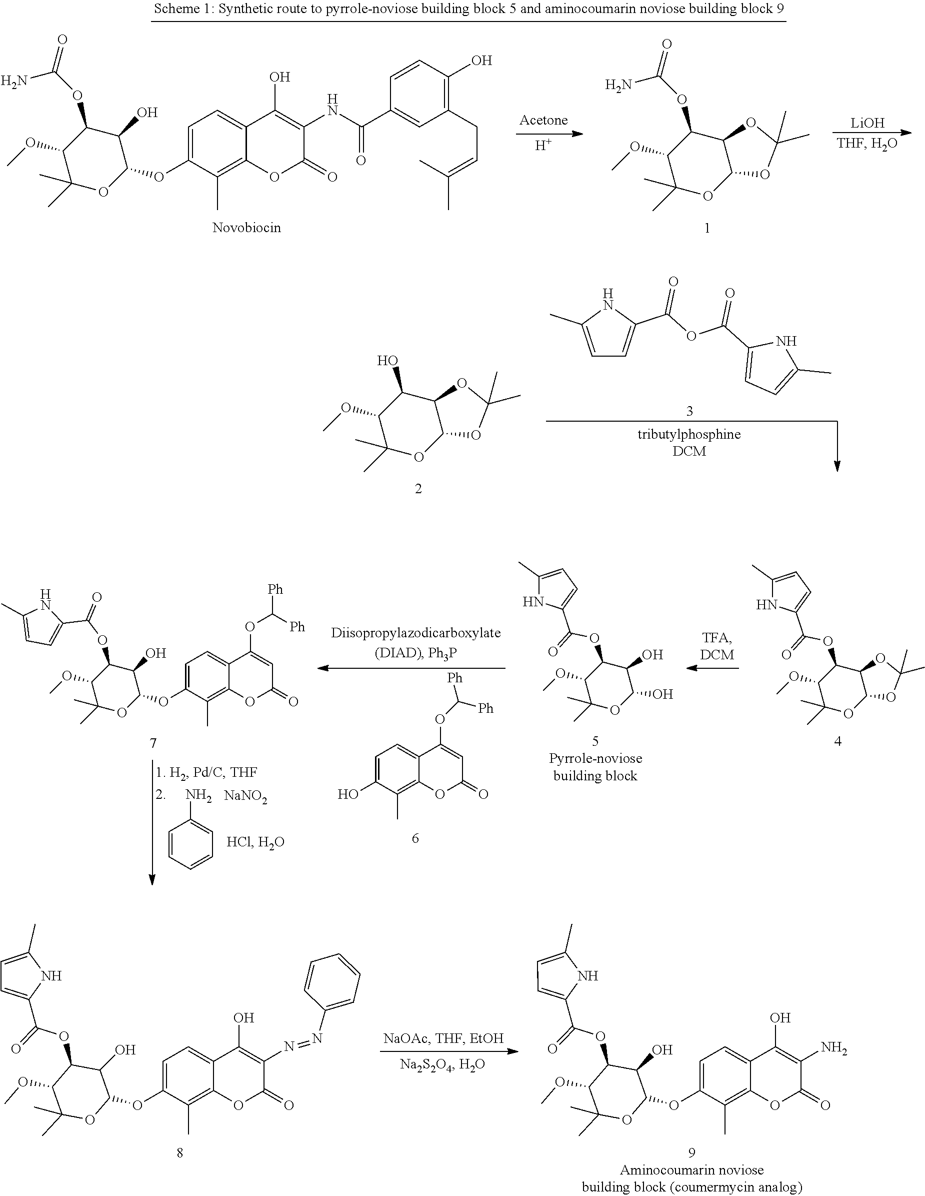

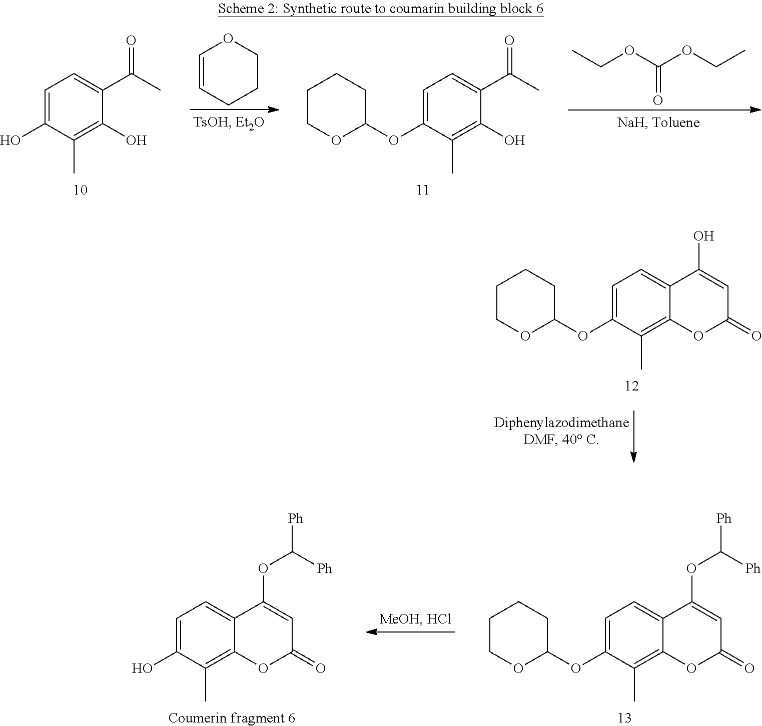

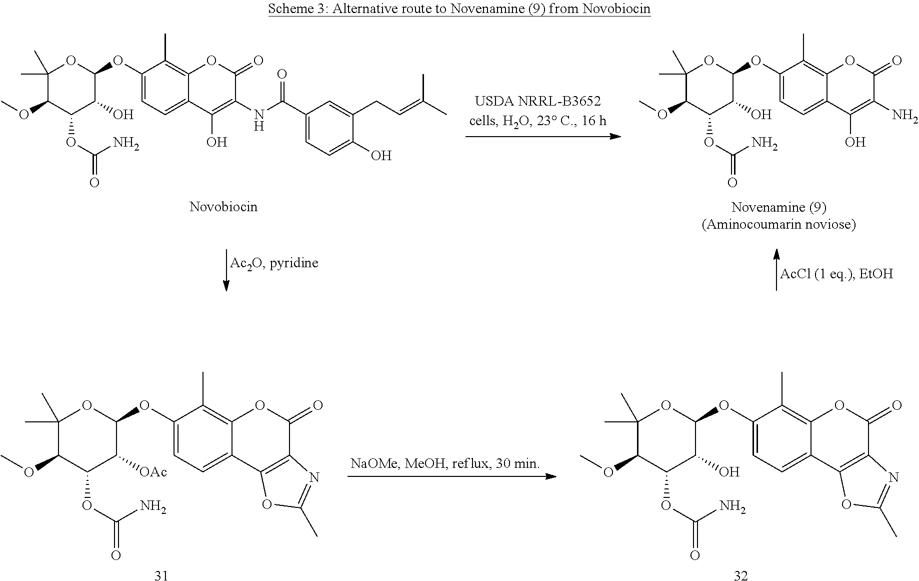 [SMILES: CO[C@@H]1[C@@H](OC(N)=O)[C@@H](O)[C@H](Oc2ccc3c(O)c(NC(=O)c4ccc(O)c(CC=C(C)C)c4)c(=O)oc3c2C)OC1(C)C ; CO[C@@H]1[C@@H](OC(N)=O)[C@@H](O)[C@H](Oc2ccc3c(O)c(N)c(=O)oc3c2C)OC1(C)C ; CO[C@@H]1[C@@H](OC(N)=O)[C@@H](OC(C)=O)[C@H](Oc2ccc3c(oc(=O)c4nc(C)oc43)c2C)OC1(C)C ; CO[C@@H]1[C@@H](OC(N)=O)[C@@H](O)[C@H](Oc2ccc3c(oc(=O)c4nc(C)oc43)c2C)OC1(C)C ; "31" ; "32"]

Novenamine (9) is known to be acylated via its coumarin amino group to provide amides (Scheme 3). Using HATU amide coupling conditions an N-protected benzoic acid of type 33 provides the benzamide 34. The C-3′ carbamate group of 34 is removed by treatment with sodium hydroxide to provide the free C-3′ alcohol 35 (Scheme 3) using conditions for similar noviose benzamides. Selective acylation of the C-3′ alcohol with 5-methyl-1H-pyrrole-2-carboxylic anhydride (3) (3) provides a key intermediate 36 possessing the pyrrole-noviose, coumarin and benzamide groups (present in coumermycin) (Scheme 3). Unmasking of the protected amine group of 36 provides an aniline of type 37. This aniline is coupled to an E3-ligase ligand conjugated to a linker with a terminal carboxylic acid, as represented by an acid 38 (see WO2020200291) which incorporates a thalidomide-like cereblon ligand to provide the helicase degrader amide of type 39.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various aspects, the terms “consisting essentially of” and “consisting of” can be used in place of “comprising” and “including” to provide for more specific aspects of the disclosure and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound of Formula I 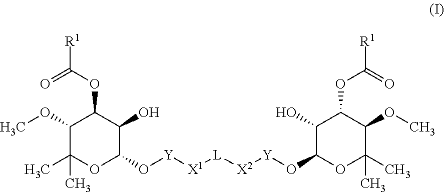
or a pharmaceutically acceptable salt thereof;
wherein:
R¹ is independently selected at each occurrence from monocyclic or bicyclic heteroaryl and monocyclic or bicyclic heterocycloalkyl, wherein each R¹ optionally includes at least one ring nitrogen atom substituted with R⁷ as allowed by valency, and wherein each R¹ is optionally substituted with 1, 2, 3, or 4 groups selected from R⁸ as allowed by valency;
Y is independently selected at each occurrence from a bond, bicyclic aryl, or bicyclic heteroaryl, wherein each Y is optionally substituted with 1, 2, 3, or 4 groups independently selected from R² as allowed by valency;
X¹ is selected from a bond or —NR^{a}—;
L is selected from a bond or L¹-L²-L³-L⁴-L⁵-L⁶-L⁷-;
L¹, L², L³, L⁴, L⁵, L⁶, and L⁷ are independently selected from:
a) a bond; 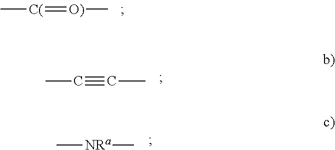
e) cycloalkyl optionally substituted with 1, 2, 3, or 4 substituents independently selected from R³;
f) heterocycloalkyl optionally substituted with 1, 2, 3, or 4 substituents independently selected from R⁴;
g) aryl optionally substituted with 1, 2, 3, or 4 groups independently selected from R⁵;
h) heteroaryl optionally substituted with 1, 2, 3, or 4 groups independently selected from R⁶; 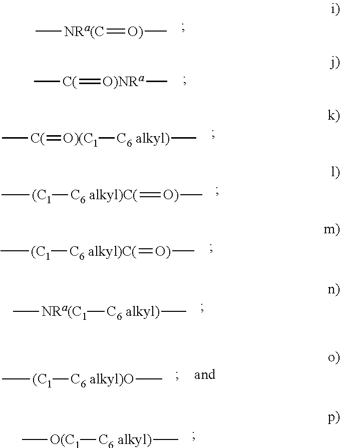
X² is selected from a bond, —NR^{a}—, —O—, —C≡C—, —C(═O)—, —S(═O)₂—, —C(═O)NR^{a}—, —NR^{a}C(═O)—, —S(═O)₂NR^{a}—, —NR^{a}S(═O)₂—, and —NR^{a} (C═O)NR^{b}—;
R² is selected at each occurrence from halogen, hydroxy, nitro, —NHR^{a}, C₁-C₆ alkyl, C₁-C₆ haloalkyl, aryl, and oxo;
R³ and R⁴ are independently selected at each occurrence from halogen, hydroxy, —NHR^{a}, C₁-C₆ alkyl, C₁-C₆ haloalkyl and oxo;
R⁵ and R⁶ are independently selected at each occurrence from halogen, hydroxy, nitro, —NHR^{a}, C₁-C₆ alkyl, C₁-C₆ haloalkyl, and aryl;
R⁷ is selected from: hydrogen; C₁-C₄ alkyl; C₂-C₆ alkenyl; C₂-C₆ alkynyl; C₃-C₆ cycloalkyl; C₁-C₆ alkoxy; and (C₀-C₆ alkyl) (aryl) which is optionally substituted with halogen, C₁-C₆ alkyl, or C₁-C₆ alkoxy;
R⁸ is selected from: hydrogen; halogen; hydroxy; —NR^{a}R^{b}; nitro; C₁-C₆ alkyl; C₁-C₆ haloalkyl; C₃-C₆ cycloalkyl; C₃-C₆ heterocycloalkyl; C₂-C₆ alkenyl; C₂-C₆ alkynyl; C₁-C₆ alkoxy; (C₀-C₆ alkyl) (aryl) which is optionally substituted with halogen, C₁-C₆ alkyl, or C₁-C₆ alkoxy; and (C₀-C₆ alkyl) (heteroaryl) which is optionally substituted with halogen, C₁-C₆ alkyl, or C₁-C₆ alkoxy; and
R^{a} and R^{b} are independently selected at each occurrence from hydrogen and C₁-C₆ alkyl.

2. The compound of claim 1, wherein R¹ is 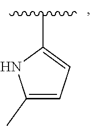 [SMILES: *c1ccc(C)[nH]1]
or
wherein R¹ is selected from:

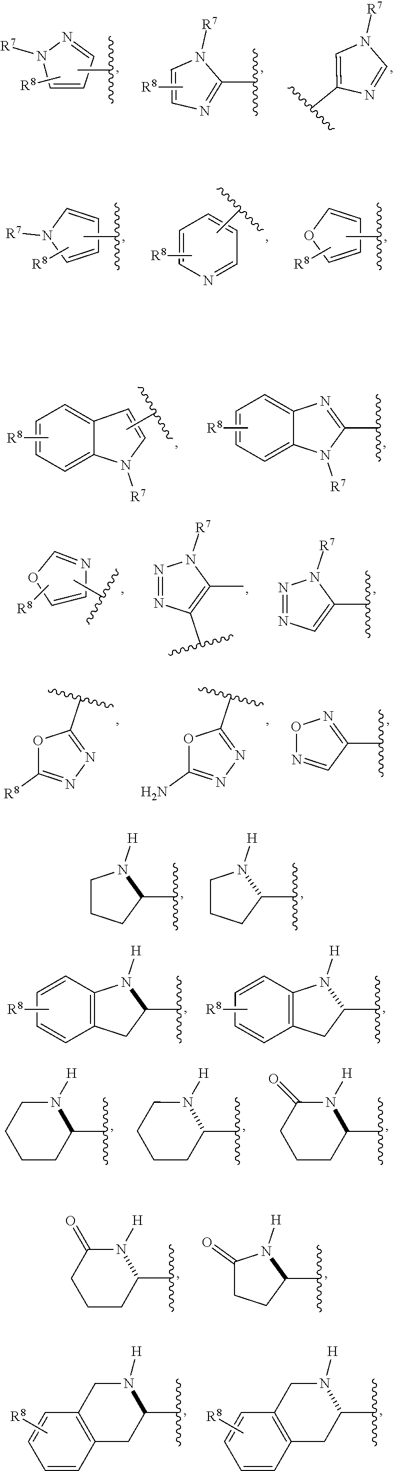

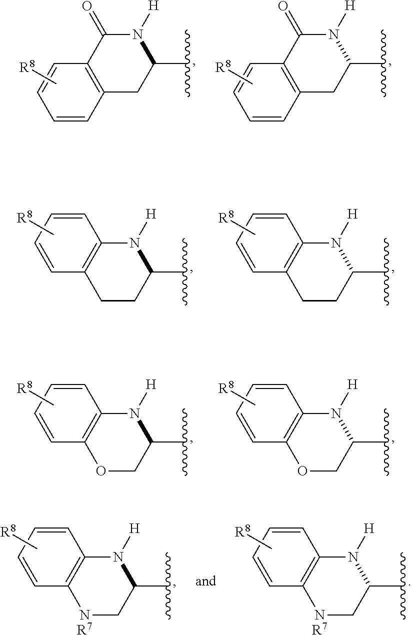

3-4. (canceled)

5. The compound of claim 1, wherein Y is a bond.

6. The compound of claim 1,
wherein Y is 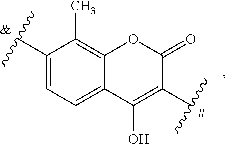 [SMILES: *c1ccc2c(O)c(*)c(=O)oc2c1C]
or
wherein Y is selected from: 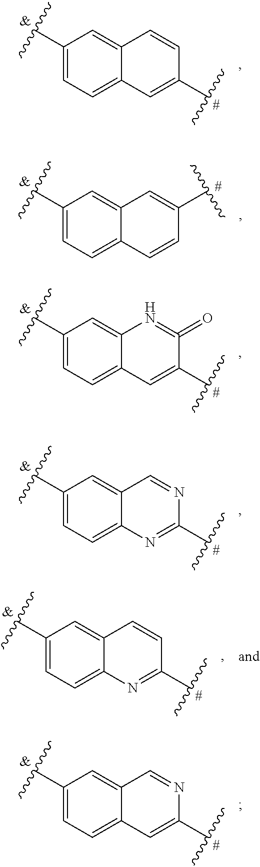
wherein & denotes the point of attachment to the neighboring oxygen atom and #denotes the point of attachment to X¹ and/or X².

7. (canceled)

8. The compound of claim 1,
wherein X¹ is —NH—.

9. The compound of claim 1, wherein L¹, L², L³, L⁴, L⁵, L⁶, and L⁷ are each independently selected from: a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O) NH—; —C(═O) CH₂—; —C(═O) (CH₂)₂—; —C(═O) (CH₂)₃—; —C(═O) (CH₂)₄—; —C(═O) (CH₂)₅—; —C(═O) (CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)₅—; —O(CH₂)₆—; 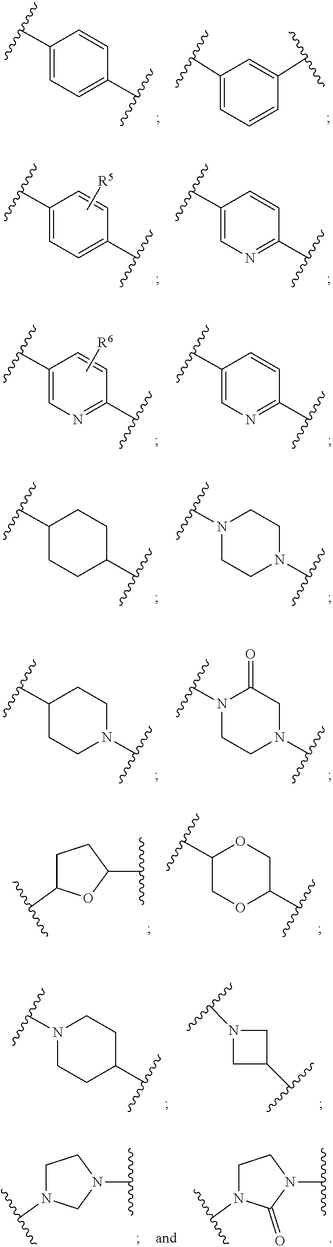
wherein L¹, L², L³, L⁴, L⁵, L⁶, and L′ are selected in such a way that: no two —C(═O)— moieties are adjected to each other; no two —O— or —NH— moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of L¹, L², L³, L⁴, L⁵, L⁶, and L⁷.

10. The compound of claim 1, wherein L is selected from: 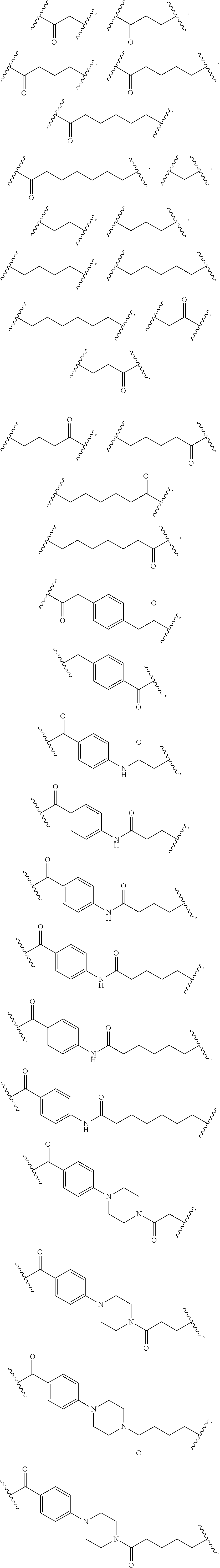 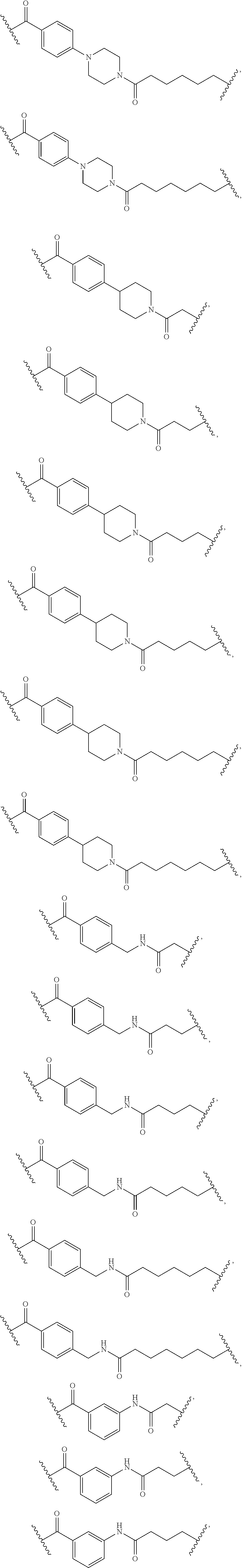 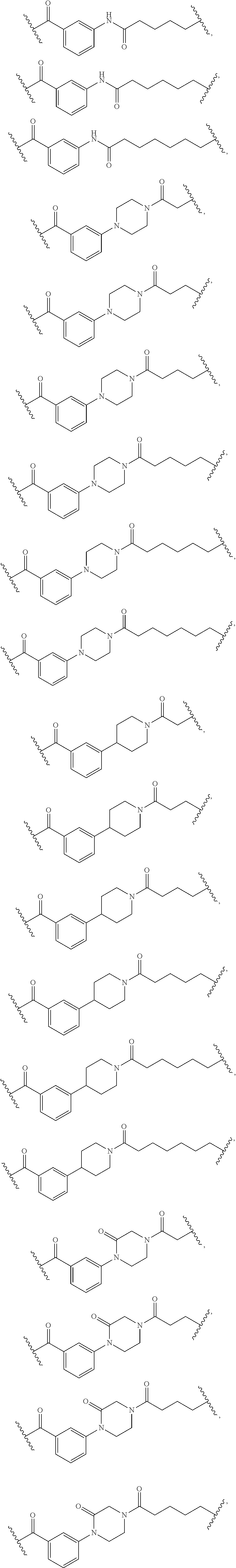 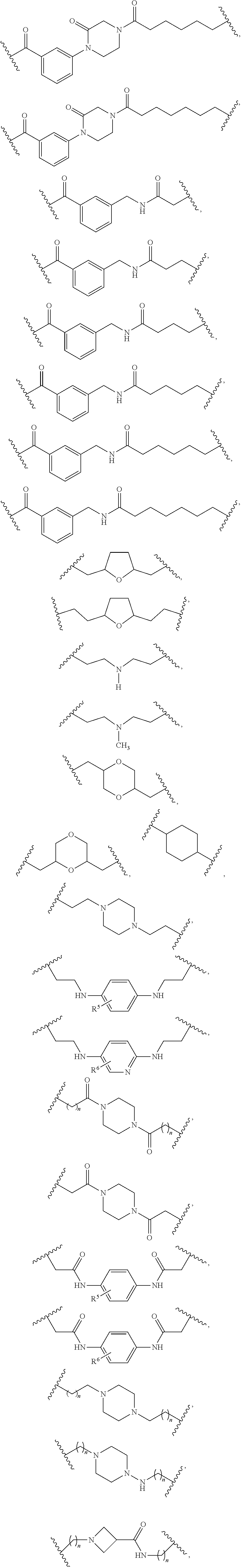 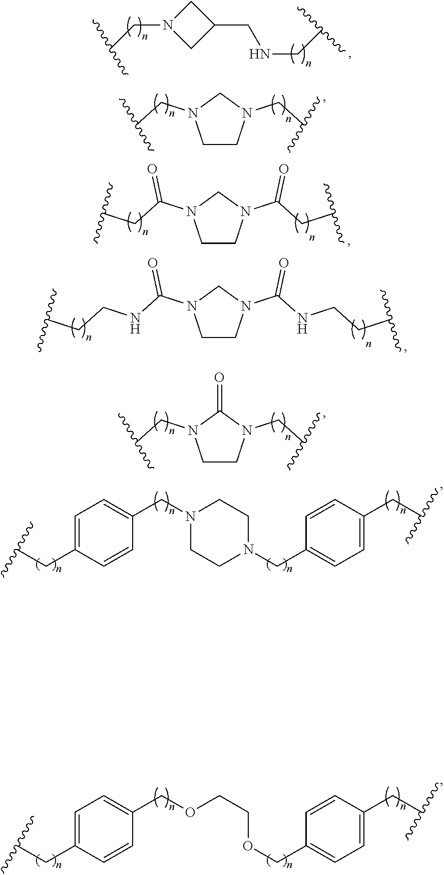 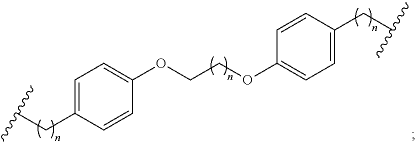
wherein n is independently selected at each occurrence from 1, 2, 3, 4, 5, and 6.

11-24. (canceled)

25. The compound of claim 1, wherein X² is a bond, —NH—, —O—, —C≡C—, —C(═O)—, —S(═O)₂—, —C(═O)NH—, —NHC(═O)—, —S(═O)₂NH—, —NHS(═O)₂—, or —NH(C═O)NH—.

26-35. (canceled)

36. A pharmaceutical composition comprising a compound of claim 1, or a pharmaceutically acceptable salt thereof, and a pharmaceutically acceptable carrier or excipient.

37. A method for treating cancer in a subject in need thereof comprising administering to the subject a therapeutically effective amount of a compound of claim 1, or a pharmaceutically acceptable salt thereof.

38. The method of claim 37, wherein the cancer is associated with or mediated by a helicase.

39-45. (canceled)

46. A method for treating cancer in a subject in need thereof comprising:
(a) determining whether the cancer is associated with elevated expression of Myc and/or elevated expression of Cyclin E; and
(b) if the cancer is determined to be associated with elevated expression of Myc and/or elevated expression of Cyclin E in (a), administering a therapeutically effective amount of a compound of claim 1, or a pharmaceutically acceptable salt thereof.

47. A method for treating cancer in a subject in need thereof, wherein the cancer has been previously determined to be associated with elevated expression of Myc and/or elevated expression of Cyclin E, the method comprising administering a therapeutically effective amount of a compound of claim 1, or a pharmaceutically acceptable salt thereof.

48. The method of claim 46, wherein the cancer is associated with overactivation of CMG helicase.

49-50. (canceled)

51. A method of treating an infection with a papillomavirus in a subject in need thereof comprising administering a therapeutically effective amount of a compound of claim 1, or a pharmaceutically acceptable salt thereof.

52-56. (canceled)

57. A method for inhibiting a helicase in a eukaryotic cell comprising contacting the cell with an effective amount of a compound of claim 1, or a pharmaceutically acceptable salt thereof.

58-61. (canceled)

62. A method for inhibiting replication of a papillomavirus in a eukaryotic cell comprising contacting the cell with an effective amount of a compound of claim 1, or a pharmaceutically acceptable salt thereof.

63-65. (canceled)

67. A method for treating cancer in a subject in need thereof comprising:
(a) determining whether the cancer harbors one or more inherited or acquired germ-line mutations; and
(b) if the cancer is determined to harbor one or more inherited or acquired germ-line mutations in (a), administering to the subject a therapeutically effective amount of a compound of claim 1, or a pharmaceutically acceptable salt thereof.

68. A method for treating cancer in a subject in need thereof, wherein the cancer has been previously determined to harbor one or more inherited or acquired germ-line mutations, the method comprising administering to the subject a therapeutically effective amount of a compound of claim 1, or a pharmaceutically acceptable salt thereof.

69-71. (canceled)

72. A method for treating cancer in a subject in need thereof comprising:
(a) administering to the subject a therapeutically effective amount of a compound of claim 1, or a pharmaceutically acceptable salt thereof; and
(b) administering an additional therapeutic agent selected from a Chk1 inhibitor, an ATR inhibitor, a Cdc7 inhibitor, and a Parp inhibitor.
